(19) United States
(12) Patent Application Publication
(10) Pub. No.: US 2023/0165958 A1
(43) Pub. Date: Jun. 1, 2023

(54) Precision Dosing Regimen

(71) Applicant: CHILDREN'S HOSPITAL MEDICAL CENTER, Cincinnati, OH (US)

(72) Inventors: Rebecca MARSH, Cincinnati, OH (US); Parinda A. MEHTA, Mason, OH (US); Alexander A. VINKS, Cincinnati, OH (US)

(73) Assignee: CHILDREN'S HOSPITAL MEDICAL CENTER, Cincinnati, OH (US)

(21) Appl. No.: 17/997,264

(22) PCT Filed: Apr. 27, 2021
(86) PCT No.: PCT/US2021/029387

§ 371 Date: Oct. 27, 2022

Related U.S. Application Data
(60) Provisional application No. 63/015,730, filed on Apr. 27, 2020.

(51) Int. Cl. A61K 39/395; A61K 35/28; A61K 9/00

(52) U.S. Cl. CPC A61K 39/3955; A61K 35/28; A61K 9/0019; A61K 2039/545

(57) ABSTRACT

The disclosure is directed to precision dosing regimens to achieve a target concentration of alemtuzumab in a subject of between about 0.15 μg/mL-about 0.6 μg/mL at day 0, or the day of a transplantation event involving allogeneic hematopoietic cells. The disclosure is also directed to methods of increasing the percentage of a patient population having a concentration of alemtuzumab of between about 0.15 μg/mL-about 0.6 μg/mL at day 0.

### TECHNICAL FIELD

The disclosure is directed to precision dosing regimens to achieve a target concentration of alemtuzumab in a subject at day 0, or the day of a transplantation event.

### BACKGROUND OF THE INVENTION

Alemtuzumab is a humanized monoclonal antibody directed against the glycoprotein CD52, expressed on the surface of the majority of mature lymphocytes and some other leukocytes. Alemtuzumab is commonly used as part of reduced-intensity conditioning (RIC) regimens for allogeneic hematopoietic cell transplantation (HCT) in pediatric patients with nonmalignant diseases.

Alemtuzumab dosing and concentrations at day 0 have previously been shown to influence transplant outcomes, including the rate of acute graft-versus-host disease (GVHD), development of mixed donor and recipient chimerism, and early immune reconstitution. Distal dosing schedules (starting day −21) can lead to non-lytic concentrations of alemtuzumab on day 0, placing patients at high risk of acute GVHD due to lack of adequate T cell depletion of the graft. Conversely, high doses of alemtuzumab or proximal and intermediate dosing schedules (starting day −8 or −14) can result in high day 0 concentrations, which are associated with decreased rates of acute GVHD but increased rates of mixed chimerism and delayed immune reconstitution. As such, a lytic concentration of alemtuzumab (>0.1-0.15 μg/mL) at day 0 is desirable to decrease the risk of acute GVHD, but the concentration should not be so high as to unacceptably increase the risk of mixed chimerism and delayed immune reconstitution. Specifically, prior analysis has found day 0 alemtuzumab concentrations of >0.6 μg/mL to be associated with high rates of mixed chimerism and delayed immune reconstitution. Therefore, an ideal therapeutic window for day 0 alemtuzumab concentrations is 0.15-0.6 μg/mL.

The pharmacokinetics of alemtuzumab is complicated with non-linear clearance and tremendous interpatient variability. Initial alemtuzumab clearance is influenced by target mediated drug disposition and dependent on the amount of CD52 antigen present. However, following the depletion of CD52 expressing leukocytes, clearance lengthens. Previous studies have explored the pharmacokinetics of alemtuzumab at standard intermediate dosing (1 mg/kg alemtuzumab divided over 5 days starting on day −14) in 17 pediatric and young adult patients with primary immunodeficiency undergoing RIC HCT. The median terminal half-life (following depletion of CD52 expressing leukocytes) of alemtuzumab in these patients was 5.2 days, and the median day 0 alemtuzumab concentration was 1.3 μg/mL (range of 0-2.6 μg/mL), which is above the upper limit of the preferred window of 0.6 μg/mL. Almost all patients had persistence of lytic concentrations of alemtuzumab beyond day 0.

Thus, there is a need to improve dosing strategies such that a greater proportion of patients end up in the ideal therapeutic concentration window of 0.15-0.6 μg/mL at day 0, or the day of a transplantation event. Such dosing strategies can minimize the risks of acute GVHD and mixed chimerism while optimizing early immune recovery.

### SUMMARY OF THE INVENTION

The disclosure provides dosing regimens, and uses thereof, to achieve a target concentration of about 0.15-0.6 μg/mL of alemtuzumab in a subject at day 0, or the day of a transplantation event. The disclosure also provides dosing regimens, and uses thereof, to achieve an increase in the number of patients in a patient population having a target concentration of about 0.15-0.6 μg/mL of alemtuzumab at day 0.

In some embodiments, provided are methods of treating a non-malignant disorder in a subject comprising transplanting allogeneic hematopoietic cells into the subject; wherein the subject has a blood concentration of alemtuzumab in a range of about 0.15 μg/mL-about 0.6 μg/mL on day 0; wherein day 0 is the day when the transplanting of the allogeneic hematopoietic cells into the subject occurs.

In some embodiments, provided are methods of treating a non-malignant disorder in a subject comprising transplanting allogeneic hematopoietic cells into the subject; wherein the subject has a blood concentration of alemtuzumab in a range of about 0.15 μg/mL-about 0.6 μg/mL on day 0;
wherein day 0 is the day, or a planned day, when the transplanting of the allogeneic hematopoietic cells into the subject occurs, the method further comprising:
administering to a subject alemtuzumab starting at day −14; wherein day −14 is two weeks or 14 days before day 0;
determining alemtuzumab concentration levels in the subject before the first dose on day −14; determining alemtuzumab concentration levels in the subject after administration of alemtuzumab on day −12; and determining alemtuzumab concentration levels in the subject daily until day 0;
modeling pharmacokinetic concentration time profiles of alemtuzumab between day −6 to day −4; wherein day −6 is six days before day 0 and day −4 is four days before day 0; and
predicting the alemtuzumab levels in the subject on day 0 using the pharmacokinetic modeling;
wherein if the alemtuzumab levels are predicted to fall below about 0.15 μg/mL on day 0, a top-up dose of alemtuzumab is given to the subject on day −3, day −2 or day −1; wherein day −3 is three days before day 0, day −2 is two days before day 0 and day −1 is one day before day 0.

### BRIEF DESCRIPTION OF THE DRAWINGS

FIG. 1 depicts an alemtuzumab dosing and concentration monitoring schedule.

FIG. 2 depicts plots of patient absolute lymphocyte counts (ALC) and Day 0 alemtuzumab concentrations with time after starting therapy. The shaded area represents the ideal therapeutic window of 0.15-0.6 μg/mL. Top-up doses are indicated by ↓.

FIG. 3 depicts a plot of observed and predicted alemtuzumab concentrations on day 0. The shaded area represents the ideal therapeutic window of 0.15-0.6 μg/mL. Patients who received a top-up dose are indicated with an asterisk (*). Patient 9 alemtuzumab concentrations were still increasing at day −6 and so simulations were not performed.

FIG. 4 depicts a plot of alemtuzumab concentrations at day 0 with precision alemtuzumab dosing (0.5-0.6 mg/kg divided over 3 days starting on day −14) and previously reported traditional intermediate alemtuzumab dosing (1 mg/kg divided over 5 days starting on day −14). Bars represent the median with interquartile range. The shaded area represents the ideal therapeutic window of 0.15-0.6 μg/mL.

FIG. 5 depicts plots of absolute CD3+ T cell, CD8+ T cell, NK cell and CD19+ B cell counts at day +100 in patients with day 0 alemtuzumab concentrations within or below the target window (i.e., <0.6 μg/mL) compared to those with day 0 alemtuzumab concentrations above the target window (i.e., >0.6 μg/mL). Bars represent mean with standard error of the mean.

FIG. 6 depicts a reduced intensity conditioning (RIC) regimen.

FIG. 7 depicts a plot of body weight vs. age distribution of study subjects and CDCNHANES database.

FIG. 8 depicts goodness-of-fit plots for the final PK model (FIGS. 8A-8D) and for the final PK-PD model (FIGS. 8E-8H). FIGS. 8A and 8E depict population prediction vs. observations. FIGS. 8B and 8F depict individual prediction vs. observations. FIGS. 8C and 8G depict conditional weighted residuals (CWRES) vs. population prediction. FIGS. 8D and 811 depict conditional weighted residuals (CWRES) vs. time after dose. Solid line, unity line (for FIGS. 8A, 8B, 8E and 8F, linear regression with a slope=1, for FIGS. 8C, 8D, 8E and 8F, line of identity showing zero). Red line, local regression line.

FIG. 9 depicts a visual predictive check of the final population PK (FIG. 9A) and PK-PD (FIG. 9B) model. Circles: observed plasma concentrations; red dashed lines: observed 10th and 90th percentile; red solid line: observed median; shaded areas, confidence intervals around the 10th, 50th and 90th percentile predictions.

FIG. 10 depicts a plot that correlates of alemtuzumab exposure with weight or age in the pilot study.

FIG. 11 depicts plots of Monte Carlo simulations of allometry-based dosing regimens.

FIG. 11A depicts simulated alemtuzumab PK profiles with a cumulative dose of 16 mg*(WT/70 kg)^{0.75}, 18 487 mg*(WT/70 kg)^{0.75}, 20 mg*(WT/70 kg)^{0.75} or 22 mg*(WT/70 kg)^{0.75} divided to three doses. FIG. 11B depicts a plot for patients who had a Day 0 concentration below 0.15 μg/mL, a top-up dose of 7 mg*(WT/70 kg)^{0.75} administered on Day −3 would bring most of patients to the exposure target range. The red lines represent predicted mean concentrations and the shaded areas indicate the 10 percentile to 90 percentile prediction intervals. The grey dashed lines shows the target of 0.15-0.6 μg/ml.

FIG. 12 depicts plots of Monte Carlo simulation of BSA-based dosing. FIG. 12A depicts simulated alemtuzumab PK profiles with a BSA-based dosing scheme. FIG. 12B depicts a plot for patients who had a Day 0 concentration below 0.15 μg/mL, a top-up dose of 3.7 kg/m² administered on Day −3 would bring most of patients to the exposure target range. The red lines represent predicted mean concentrations and the shaded areas indicate the 10 percentile to 90 percentile prediction intervals. The grey dashed lines shows the target of 0.15-0.6 μg/ml.

FIG. 13 depicts plots of Monte Carlo simulations of per kg dosing. Simulated alemtuzumab PK profiles with a per KG dosing scheme. The red lines represent predicted mean concentrations and the shaded areas indicate the 10 percentile to 90 percentile prediction intervals. The grey dashed lines shows the target of 0.15-0.6 μg/ml.

FIG. 14 depicts simulated projected alemtuzumab concentration on Day 0 across different ages. Per kg dosing would result in uneven alemtuzumab exposure across different age spectra, whereas BSA- or allometry-based dosing showed overall similar exposure levels in different age groups.

FIG. 15 depicts proposed alemtuzumab precision dosing imbedded with Bayesian estimation and illustrates the proposed alemtuzumab precision dosing strategy. Following initial drug administrations on Day −14 to Day −12, alemtuzumab concentrations are measured and used for Bayesian estimation on Day −5 for a possible additional dose selection to achieve a target concentration of 0.15-0.6 μg/ml. The dashed line represents the model predicted alemtuzumab PK profile in a typical subject. The red dots represent measured alemtuzumab concentrations. The solid line represents the Bayesian estimated individual PK profile including the predicted increase in concentration after the additional dose. Note that if no top-up dose was given, the projected Day 0 concentration would be below the target.

FIG. 16 depicts predicted dose levels using BSA-based dosing calculation, allometry-based dosing calculation and per kg based dosing calculation approaches. FIG. 16A depicts the alemtuzumab initial dose calculated by different dosing algorithms. FIG. 16B depicts BSA- and allometry-based dosing leads to higher per body weight (kg) doses in infants compared to older children and young adults.

### DETAILED DESCRIPTION OF ILLUSTRATIVE EMBODIMENTS

The disclosure may be more fully appreciated by reference to the following description, including the following definitions and examples. Certain features of the disclosed processes are described herein in the context of separate aspects, may also be provided in combination in a single aspect. Alternatively, various features of the disclosed processes that are, for brevity, described in the context of a single aspect, may also be provided separately or in any sub-combination.

In an aspect, the disclosure is directed to methods of treating a non-malignant disorder in a subject comprising transplanting allogeneic hematopoietic cells into the subject; wherein the subject has a blood concentration of alemtuzumab in a range of about 0.15 μg/mL-about 0.6 μg/mL on day 0; wherein day 0 is the day, or a planned day, when the transplanting of the allogeneic hematopoietic cells into the subject occurs.

In another aspect, the disclosure is directed to methods of treating a non-malignant disorder in a subject comprising transplanting allogeneic hematopoietic cells into the subject; wherein the subject has a blood concentration of alemtuzumab in a range of about 0.15 μg/mL-about 0.6 μg/mL on day 0;
wherein day 0 is the day, or a planned day, when the transplanting of the allogeneic hematopoietic cells into the subject occurs, the method further comprising:
administering to a subject alemtuzumab starting at day −14; wherein day −14 is two weeks or 14 days before day 0;
determining alemtuzumab concentration levels in the subject before the first dose on day −14; determining alemtuzumab concentration levels in the subject after administration of alemtuzumab on day −12; and determining alemtuzumab concentration levels in the subject daily until day 0;
modeling pharmacokinetic concentration time profiles of alemtuzumab between day −6 to day −4; wherein day −6 is six days before day 0 and day −4 is four days before day 0; and
predicting the alemtuzumab levels in the subject on day 0 using the pharmacokinetic modeling;
wherein if the alemtuzumab levels are predicted to fall below about 0.15 μg/mL on day 0, a top-up dose of alemtuzumab is given to the subject on day −3, day −2 or day −1; wherein day −3 is three days before day 0, day −2 is two days before day 0 and day −1 is one day before day 0.

In an aspect, the disclosure is directed to methods of increasing the percentage of a patient population having a blood concentration of alemtuzumab in a range of about 0.15 μg/mL-about 0.6 μg/mL on day 0; wherein the patient population is in need of treatment for a non-malignant disorder.

In some embodiments of the aspects described herein, the day 0 concentration of alemtuzumab in the patient is between about 0.15 μg/mL-about 0.6 μg/mL; or the day 0 concentration of alemtuzumab in the patient is between about 0.15 μg/mL-about 0.2 μg/mL; or the day 0 concentration of alemtuzumab in the patient is between about 0.2 μg/mL-about 0.25 μg/mL; or the day 0 concentration of alemtuzumab in the patient is between about 0.25 μg/mL-about 0.3 μg/mL; or the day 0 concentration of alemtuzumab in the patient is between about 0.3 μg/mL-about 0.35 μg/mL; or the day 0 concentration of alemtuzumab in alemtuzumab in the patient is between about 0.35 μg/mL-about 0.4 μg/mL; or the day 0 concentration of alemtuzumab in the patient is between about 0.4 μg/mL-about 0.45 μg/mL; or the day 0 concentration of alemtuzumab in the patient is between about 0.45 μg/mL-about 0.5 μg/mL; or the day 0 concentration of alemtuzumab in the patient is between about 0.5 μg/mL-about 0.55 μg/mL; or the day 0 concentration of alemtuzumab in the patient is between about 0.55 μg/mL-about 0.6 μg/mL.

In some embodiments of the aspects described herein, the methods comprise administering to the subject alemtuzumab starting at a day between day −22 and day −8; wherein day −22 is twenty-two days before day 0 and day −8 is eight days before day 0.

In some embodiments of the aspects described herein, the methods comprise administering to the subject alemtuzumab starting at a day between day −20 and day −9; wherein day −20 is twenty days before day 0 and day −9 is nine days before day 0.

In some embodiments of the aspects described herein, the methods comprise administering to the subject alemtuzumab starting at a day between day −18 and day −10; wherein day −18 is eighteen days before day 0 and day −10 is ten days before day 0.

In some embodiments of the aspects described herein, the methods comprise administering to the subject alemtuzumab starting at a day between day −16 and day −12; wherein day −16 is sixteen days before day 0 and day −12 is twelve days before day 0.

In some embodiments of the aspects described herein, the methods comprise administering to the subject alemtuzumab starting at day −14; wherein day −14 is two weeks or 14 days before day 0.

In some embodiments of the aspects described herein, the methods comprise administering alemtuzumab to the subject over two, three, four or five days starting at a day between day −22 and day −8.

In some embodiments of the aspects described herein, the methods comprise administering alemtuzumab to the subject over two days starting at a day between day −22 and day −8.

In some embodiments, the two days are day −22 and day −21. In some embodiments, the two days are day −21 and day −20. In some embodiments, the two days are day −20 and day −19. In some embodiments, the two days are day −19 and day −18. In some embodiments, the two days are day −18 and day −17. In some embodiments, the two days are day −17 and day −16. In some embodiments, the two days are day −16 and day −15. In some embodiments, the two days are day −15 and day −14. In some embodiments, the two days are day −14 and day −13. In some embodiments, the two days are day −13 and day −12. In some embodiments, the two days are day −12 and day −11. In some embodiments, the two days are day −11 and day −10. In some embodiments, the two days are day −10 and day −9. In some embodiments, the two days are day −9 and day −8.

In some embodiments of the aspects described herein, the methods comprise administering alemtuzumab to the subject over three days starting at a day between day −22 and day −8.

In some embodiments, the three days are day −22, day −21 and day −20. In some embodiments, the three days are day −21, day −20 and day −19. In some embodiments, the three days are day −20, day −19 and day −18. In some embodiments, the three days are day −19, day −18 and day −17. In some embodiments, the three days are day −18, day −17 and day −16. In some embodiments, the three days are day −17, day −16 and day −15. In some embodiments, the three days are day −16, day −15 and day −14. In some embodiments, the three days are day −15, day −14 and day −13. In some embodiments, the three days are day −14, day −13 and day −12. In some embodiments, the three days are day −13, day −12 and day −11. In some embodiments, the three days are day −12, day −11 and day −10. In some embodiments, the three days are day −11, day −10 and day −9. In some embodiments, the three days are day −10, day −9 and day −8.

In some embodiments of the aspects described herein, the methods comprise administering alemtuzumab to the subject over four days starting at a day between day −22 and day −8.

In some embodiments, the four days are day −22, day −21, day −20 and day −19. In some embodiments, the four days are day −21, day −20, day −19 and day −18. In some embodiments, the four days are day −20, day −19, day −18 and day −17. In some embodiments, the four days are day −19, day −18, day −17 and day −16. In some embodiments, the four days are day −18, day −17, day −16 and day −15. In some embodiments, the four days are day −17, day −16, day −15 and day −14. In some embodiments, the four days are day −16, day −15, day −14 and day −13. In some embodiments, the four days are day −15, day −14, day −13 and day −12. In some embodiments, the four days are day −14, day −13, day −12 and day −11. In some embodiments, the four days are day −13, day −12, day −11 and day −10. In some embodiments, the four days are day −12, day −11, day −10 and day −9. In some embodiments, the four days are day −11, day −10, day −9 and day −8.

In some embodiments of the aspects described herein, the methods comprise administering alemtuzumab to the subject over five days starting at a day between day −22 and day −8.

In some embodiments, the five days are day −22, day −21, day −20, day −19 and day −18.

In some embodiments, the five days are day −21, day −20, day −19, day −18 and day −17. In some embodiments, the five days are day −20, day −19, day −18, day −17 and day −16. In some embodiments, the five days are day −19, day −18, day −17, day −16 and day −15. In some embodiments, the five days are day −18, day −17, day −16, day −15 and day −14. In some embodiments, the five days are day −17, day −16, day −15, day −14 and day −13. In some embodiments, the five days are day −16, day −15, day −14, day −13 and day −12. In some embodiments, the five days are day −15, day −14, day −13, day −12 and day −11. In some embodiments, the five days are day −14, day −13, day −12, day −11 and day −10. In some embodiments, the five days are day −13, day −12, day −11, day −10 and day −9. In some embodiments, the five days are day −12, day −11, day −10, day −9 and day −8.

In some embodiments of the aspects described herein, the methods comprise administering to the subject between about 0.45 mg/kg-about 0.65 mg/kg alemtuzumab over the two, three, four or five days. In some embodiments of the aspects described herein, the methods comprise administering to the subject between about 0.45 mg/kg-about 0.65 mg/kg alemtuzumab over the two days. In some embodiments of the aspects described herein, the methods comprise administering to the subject between about 0.45 mg/kg-about 0.65 mg/kg alemtuzumab over the three days. In some embodiments of the aspects described herein, the methods comprise administering to the subject between about 0.45 mg/kg-about 0.65 mg/kg alemtuzumab over the four days. In some embodiments of the aspects described herein, the methods comprise administering to the subject between about 0.45 mg/kg-about 0.65 mg/kg alemtuzumab over the five days.

In some embodiments, the amount of alemtuzumab administered to the subject over the two, three, four or five days is between about 0.5 mg/kg-about 0.6 mg/kg alemtuzumab. In some embodiments of the aspects described herein, the methods comprise administering to the subject between about 0.5 mg/kg-about 0.6 mg/kg alemtuzumab over the two days. In some embodiments of the aspects described herein, the methods comprise administering to the subject between about 0.5 mg/kg-about 0.6 mg/kg alemtuzumab over the three days. In some embodiments of the aspects described herein, the methods comprise administering to the subject between about 0.5 mg/kg-about 0.6 mg/kg alemtuzumab over the four days. In some embodiments of the aspects described herein, the methods comprise administering to the subject between about 0.5 mg/kg-about 0.6 mg/kg alemtuzumab over the five days.

In some embodiments of the aspects described herein, the methods comprise administering to the subject between about 8 mg/m²-about 12 mg/m² alemtuzumab over the two, three, four or five days. In some embodiments of the aspects described herein, the methods comprise administering to the subject between about 8 mg/m²-about 12 mg/m² alemtuzumab over the two days. In some embodiments of the aspects described herein, the methods comprise administering to the subject between about 8 mg/m²-about 12 mg/m² alemtuzumab over the three days. In some embodiments of the aspects described herein, the methods comprise administering to the subject between about 8 mg/m²-about 12 mg/m² alemtuzumab over the four days. In some embodiments of the aspects described herein, the methods comprise administering to the subject between about 8 mg/m²-about 12 mg/m² alemtuzumab over the five days.

In some embodiments, the amount of alemtuzumab administered to the subject over the two, three, four or five days is about 10 mg/m² alemtuzumab. In some embodiments of the aspects described herein, the methods comprise administering to the subject about 10 mg/m² alemtuzumab over the two days. In some embodiments of the aspects described herein, the methods comprise administering to the subject about 10 mg/m² alemtuzumab over the three days. In some embodiments of the aspects described herein, the methods comprise administering to the subject about 10 mg/m² alemtuzumab over the four days. In some embodiments of the aspects described herein, the methods comprise administering to the subject about 10 mg/m² alemtuzumab over the five days.

In some embodiments of the aspects described herein, if the subject weighs less than about 15 kg, the subject is administered about 0.2 mg/kg/dose of alemtuzumab on each of the days. In some embodiments of the aspects described herein, if the subject weighs less than about 15 kg, the subject is administered about 0.2 mg/kg/dose of alemtuzumab on each of the three days. In some embodiments of the aspects described herein, if the subject weighs less than about 15 kg, the subject is administered about 0.2 mg/kg/dose of alemtuzumab on each of the three days starting at day −14.

In some embodiments, if the subject weighs more than about 15 kg, the subject is administered a test dose of about 3 mg of alemtuzumab. In some embodiments, if the subject weighs more than about 15 kg, the subject is administered a test dose of about 3 mg of alemtuzumab on day −14.

In some embodiments, if the subject weighs more than about 15 kg, the subject is administered, after the test dose, about a 0.2 mg/kg/dose to 0.3 mg/kg/dose of alemtuzumab. In some embodiments, if the subject weighs more than about 15 kg, the subject is administered, after the test dose, about a 0.2 mg/kg/dose to 0.3 mg/kg/dose of alemtuzumab on day −13 and about a 0.2 mg/kg/dose to 0.3 mg/kg/dose on day −12.

In some embodiments, if the subject weighs more than about 15 kg, the subject is administered about 0.23 mg/kg/dose of alemtuzumab. In some embodiments, if the subject weighs more than about 15 kg, the subject is administered about 0.23 mg/kg/dose of alemtuzumab on day −13 and on day −12.

In some embodiments of the aspects described herein, the methods further comprise determining alemtuzumab concentration levels in the subject before the first dose of alemtuzumab on the day between day −22 and day −8; and determining alemtuzumab concentration levels in the subject on a day after the first dose; and determining alemtuzumab concentration levels in the subject daily until day 0.

In some embodiments, a day after the first dose is day −21; or day −20; or day −19; or day −18; or day −17; or day −16; or day −15; or day −14; or day −13; or day −12; or day −11; or day −10; or day −9; or day −8; or day −7. In some embodiments, the first dose is on day −14 and a day after the first dose is day −12.

In some embodiments of the aspects described herein, the methods further comprise determining alemtuzumab concentration levels in the subject before the first dose on a day between day −22 and day −13; determining alemtuzumab concentration levels in the subject after administration of alemtuzumab on day −12; and determining alemtuzumab concentration levels in the subject daily until day 0.

In some embodiments of the aspects described herein, the methods further comprise determining alemtuzumab concentration levels in the subject before the first dose on day −14; determining alemtuzumab concentration levels in the subject after administration of alemtuzumab on day −12; and determining alemtuzumab concentration levels in the subject daily until day 0.

In some embodiments of the aspects described herein, alemtuzumab concentration levels in the subject are determined 8 hours and 24 hours after administration of alemtuzumab on day −12.

In some embodiments of the aspects described herein, the alemtuzumab concentration levels are measured using flow cytometry.

In some embodiments of the aspects described herein, the methods further comprise carrying out pharmacokinetic modeling between day −6 to day −4 to predict day 0 levels of alemtuzumab concentration in the subject; wherein day −6 is six days before day 0 and day −4 is four days before day 0. In some embodiments, the pharmacokinetic modeling is carried out on day −5; wherein day −5 is five days before day 0. In some embodiments, the pharmacokinetic modeling is carried out on day −6. In some embodiments, the pharmacokinetic modeling is carried out on day −4.

In some embodiments of the aspects described herein, if pharmacokinetic modeling predicts the alemtuzumab levels to fall below about 0.15 μg/mL in the subject on day 0, a top-up dose of alemtuzumab is given to the subject day −3, day −2 or day −1; wherein day −3 is three days before day 0, day −2 is days before day 0 and day −1 is one day before day 0. In some embodiments, the top-up dose is given to the subject on day −3. In some embodiments, the top-up dose is given to the subject on day −2. In some embodiments, the top-up dose is given to the subject on day −1.

In some embodiments of the aspects described herein, the pharmacokinetic modeling is based on Bayesian analysis or estimation.

In some embodiments of the aspects described herein, the top-up dose is tailored for each subject and the individualized top-up dose is determined using Bayesian analysis or estimation.

In some embodiments of the aspects described herein, the top-up dose comprises about 0.2 mg/m² to about 5.0 mg/m² of alemtuzumab.

In some embodiments, the top-up dose comprises about 0.2 mg/m² to about 0.5 mg/m² of alemtuzumab; or about 0.5 mg/m² to about 1.0 mg/m² of alemtuzumab; or about 1.0 mg/m² to about 1.5 mg/m² of alemtuzumab; or about 1.5 mg/m² to about 2.0 mg/m² of alemtuzumab; or about 2.0 mg/m² to about 2.5 mg/m² of alemtuzumab; or about 2.5 mg/m² to about 3.0 mg/m² of alemtuzumab; about 3.0 mg/m² to about 3.5 mg/m² of alemtuzumab; or about 3.5 mg/m² to about 4.0 mg/m² of alemtuzumab; about 4.0 mg/m² to about 4.5 mg/m² of alemtuzumab; or about 4.5 mg/m² to about 5.0 mg/m² of alemtuzumab.

In some embodiments, the top-up dose comprises about 3.5 mg/m² to about 4.0 mg/m² of alemtuzumab. In some embodiments, the top-up dose comprises about 3.7 mg/m² of alemtuzumab.

In some embodiments of the aspects described herein, the methods further comprise administering a chemotherapeutic agent to the subject. In some embodiments, the chemotherapeutic agent is selected from fludarabine, melphalan, busulfan, treosulfan, thiotepa, cyclophosphamide and combinations thereof. In some embodiments, the chemotherapeutic agent is fludarabine. In some embodiments, the chemotherapeutic agent is melphalan. In some embodiments, the chemotherapeutic agent is busulfan. In some embodiments, the chemotherapeutic agent is treosulfan. In some embodiments, the chemotherapeutic agent is thiotepa. In some embodiments, the chemotherapeutic agent is cyclophosphamide. In some embodiments, the chemotherapeutic agent is a combinations of fludarabine and malphalan.

In some embodiments of the aspects described herein, the methods further comprise administering between about 125 mg/m²-about 200 mg/m² of fludarabine to subjects weighing at least about 10 kg; or administering between about 3 to about 7 mg/kg of fludarabine to subjects weighing less than about 10 kg.

In some embodiments of the aspects described herein, if the subject weighs at least about 10 kg, the subject is administered between about 125 mg/m²-about 200 mg/m² of fludarabine; or between about 125 mg/m²-about 130 mg/m² of fludarabine; or between about 130 mg/m²-about 135 mg/m² of fludarabine; or between about 135 mg/m²-about 140 mg/m² of fludarabine; or between about 140 mg/m²-about 145 mg/m² of fludarabine; or between about 145 mg/m²-about 150 mg/m² of fludarabine; or between about 150 mg/m²-about 155 mg/m² of fludarabine; or between about 155 mg/m²-about 160 mg/m² of fludarabine; or between about 160 mg/m²-about 165 mg/m² of fludarabine; or between about 165 mg/m²-about 170 mg/m² of fludarabine; or between about 170 mg/m²-about 175 mg/m² of fludarabine; or between about 175 mg/m²-about 180 mg/m² of fludarabine; or between about 180 mg/m²-about 185 mg/m² of fludarabine; or between about 185 mg/m²-about 190 mg/m² of fludarabine; or between about 190 mg/m²-about 195 mg/m² of fludarabine; or between about 195 mg/m²-about 200 mg/m² of fludarabine. In some embodiments, the methods further comprise administering about 150 mg/m² of fludarabine to subjects weighing at least about 10 kg.

In some embodiments of the aspects described herein, if the subject weighs less than about 10 kg, the subject is administered between about 3 to about 7 mg/kg; or between about 3 mg/m²-about 3.5 mg/m² of fludarabine; or between about 3.5 mg/m²-about 4 mg/m² of fludarabine; or between about 4 mg/m²-about 4.5 mg/m² of fludarabine; or between about 4.5 mg/m²-about 5 mg/m² of fludarabine; or between about 5 mg/m²-about 5.5 mg/m² of fludarabine; or between about 5.5 mg/m²-about 6 mg/m² of fludarabine; or between about 6 mg/m²-about 6.5 mg/m² of fludarabine; or between about 6.5 mg/m²-about 7 mg/m² of fludarabine. In some embodiments, the methods further comprise administering about 5 mg/m² of fludarabine to subjects weighing at least about 10 kg.

In some embodiments of the aspects described herein, the fludarabine is administered to the subject over 5 days. In some embodiments, the 5 days are day −8, day −7, day −6, day −5 and day −4; wherein day −8 is eight days before day 0, day −7 is seven days before day 0, day −6 is six days before day 0, day −5 is five days before day 0 and day −4 is four days before day 0.

In some embodiments of the aspects described herein, the methods further comprise administering between about 115 mg/m²-about 165 mg/m² of melphalan to subjects weighing at least about 10 kg; or administering between about 3 mg/kg to about 7 mg/kg of melphalan to subjects weighing less than about 10 kg.

In some embodiments of the aspects described herein, if the subject weighs at least about 10 kg, the subject is administered between about 115 mg/m²-about 165 mg/m² of melphalan; or between about 115 mg/m²-about 120 mg/m² of melphalan; or between about 120 mg/m²-about 125 mg/m² of melphalan; or between about 125 mg/m²-about 130 mg/m² of melphalan; or between about 130 mg/m²-about 135 mg/m² of melphalan; or between about 135 mg/m²-about 140 mg/m² of melphalan; or between about 140 mg/m²-about 145 mg/m² of melphalan; or between about 145 mg/m²-about 150 mg/m² of melphalan; or between about 150 mg/m²-about 155 mg/m² of melphalan; or between about 155 mg/m²-about 160 mg/m² of melphalan; or between about 160 mg/m²-about 165 mg/m² of melphalan. In some embodiments, the methods further comprise administering about 140 mg/m² of melphalan to subjects weighing at least about 10 kg.

In some embodiments of the aspects described herein, if the subject weighs less than about 10 kg, the subject is administered between about 3 mg/kg to about 7 mg/kg of melphalan; or between about 3 mg/m²-about 3.5 mg/m² of melphalan; or between about 3.5 mg/m²-about 4 mg/m² of melphalan; or between about 4 mg/m²-about 4.5 mg/m² of melphalan; or between about 4.5 mg/m²-about 5 mg/m² of melphalan; or between about 5 mg/m²-about 5.5 mg/m² of melphalan; or between about 5.5 mg/m²-about 6 mg/m² of melphalan; or between about 6 mg/m²-about 6.5 mg/m² of melphalan; or between about 6.5 mg/m²-about 7 mg/m² of melphalan. In some embodiments, the methods further comprise administering about 4.7 mg/m² of melphalan to subjects weighing at least about 10 kg.

In some embodiments of the aspects described herein, the melphalan is administered to the subject on day −3 or day −1. In some embodiments, the melphalan is administered to the subject on day −3. In some embodiments, the melphalan is administered to the subject on day −1.

In some embodiments of the aspects described herein, the alemtuzumab is administered intravenously or subcutaneously to the subject. In some embodiments, the alemtuzumab is administered subcutaneously to the subject. In some embodiments, the alemtuzumab is administered intravenously to the subject.

In some embodiments of the aspects described herein, the patient or subject is a pediatric patient or a young adult patient. In some embodiments, the patient or subject is a pediatric patient. In some embodiments, the patient or subject is a young adult patient.

In some embodiments of the aspects described herein, the patient or subject is afflicted with the non-malignant disorder. In some embodiments, the patient or subject afflicted with the non-malignant disorder is a pediatric patient or a young adult patient.

In some embodiments of the aspects described herein, the non-malignant disorder is selected from immunodeficiencies, bone-marrow failure syndromes, inborn errors of metabolism (IEM) and hemoglobinopathies. In some embodiments, the non-malignant disorder is immunodeficiencies. In some embodiments, the non-malignant disorder is bone-marrow failure syndromes. In some embodiments, the non-malignant disorder is inborn errors of metabolism (IEM). In some embodiments, the non-malignant disorder is hemoglobinopathies.

### Definitions

As used herein, the term “day 0” or “day zero” is taken to mean the day or the planned day when the transplanting of the allogeneic hematopoietic cells into a subject occurs, or taken to mean the day or planned day of a allogeneic hematopoietic cell transplantation event.

As used herein, the term “planned day” is taken to mean the day on which the hematopoietic cell infusion is planned to occur. Unforeseen circumstances such as travel time required for a hematopoietic cell product to arrive at a stem cell transplant center may delay the actual infusion of the product by 1-2 days and result in administration on day +1 or day +2.

As used herein, the term “day −22” or “day −twenty-two” is taken to mean twenty-two, 22, days before day 0.

As used herein, the term “day −21” or “day −twenty-one” is taken to mean twenty-one, 21, days before day 0.

As used herein, the term “day −20” or “day −twenty” is taken to mean twenty, 20, days before day 0.

As used herein, the term “day −19” or “day −nineteen” is taken to mean nineteen, 19, days before day 0.

As used herein, the term “day −18” or “day −eighteen” is taken to mean eighteen, 18, days before day 0.

As used herein, the term “day −17” or “day −seventeen” is taken to mean seventeen, 17, days before day 0.

As used herein, the term “day −16” or “day −sixteen” is taken to mean sixteen, 16, days before day 0.

As used herein, the term “day −15” or “day −fifteen” is taken to mean fifteen, 15, days before day 0.

As used herein, the term “day −14” or “day −fourteen” is taken to mean two weeks or fourteen, 14, days before day 0.

As used herein, the term “day −13” or “day −thirteen” is taken to mean thirteen, 13, days before day 0.

As used herein, the term “day −12” or “day −twelve” is taken to mean twelve, 12, days before day 0.

As used herein, the term “day −11” or “day −eleven” is taken to mean eleven, 11, days before day 0.

As used herein, the term “day −10” or “day −ten” is taken to mean ten, 10, days before day 0.

As used herein, the term “day −9” or “day −nine” is taken to mean nine, 9, days before day 0.

As used herein, the term “day −8” or “day −eight” is taken to mean eight, 8, days before day 0.

As used herein, the term “day −7” or “day −seven” is taken to mean one week or seven, 7, days before day 0.

As used herein, the term “day −6” or “day −six” is taken to mean six, 6, days before day 0.

As used herein, the term “day −5” or “day −five” is taken to mean five, 5, days before day 0.

As used herein, the term “day −4” or “day −four” is taken to mean four, 4, days before day 0.

As used herein, the term “day −3” or “day −three” is taken to mean three, 3, days before day 0.

As used herein, the term “day −2” or “day −two” is taken to mean two, 2, days before day 0.

As used herein, the term “day −1” or “day −one” is taken to mean one, 1, day before day 0.

As used herein, the term “day +1” or “day +one” is taken to mean one, 1, day after day 0.

As used herein, the term “day +2” or “day +two” is taken to mean two, 2, days after day 0.

By the terms “treat,” “treating” or “treatment of” (or grammatically equivalent terms) it is meant that the severity of the subject's condition is reduced or at least partially improved or ameliorated and/or that some alleviation, mitigation or decrease in at least one clinical symptom is achieved and/or there is an inhibition or delay in the progression of the condition and/or prevention or delay of the onset of a disease or illness. The terms “treat,” “treating” or “treatment of” also means managing an autoimmune disease or disorder. Thus, the terms “treat,” “treating” or “treatment of” (or grammatically equivalent terms) refer to both prophylactic and therapeutic treatment regimes.

As used herein, a “sufficient amount” or “an amount sufficient to” achieve a particular result refers to an amount of an antibody or composition of the invention that is effective to produce a desired effect, which is optionally a therapeutic effect (i.e., by administration of a therapeutically effective amount). For example, a “sufficient amount” or “an amount sufficient to” can be an amount that is effective to deplete B cells.

A “therapeutically effective” amount as used herein is an amount that provides some improvement or benefit to the subject. Alternatively stated, a “therapeutically effective” amount is an amount that provides some alleviation, mitigation, and/or decrease in at least one clinical symptom. Clinical symptoms associated with the disorders that can be treated by the methods of the invention are well-known to those skilled in the art. Further, those skilled in the art will appreciate that the therapeutic effects need not be complete or curative, as long as some benefit is provided to the subject.

The following Examples are provided to illustrate aspects of the invention and are not intended to be limiting.

### EXAMPLES

### Example 1—a Novel Alemtuzumab Target Concentration Intervention Strategy

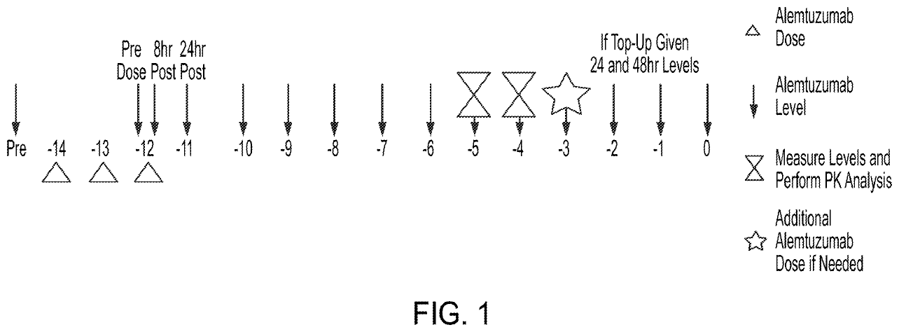

This study was approved by the institutional review board and was registered at Clinicaltrials.gov (NCT03302754). Informed consent was obtained for all patients, and informed assent was obtained for all patients 11-17 years of age. Twelve patients with nonmalignant diseases undergoing HCT with alemtuzumab, fludarabine, and melphalan containing RIC at Cincinnati Children's Hospital Medical Center were prospectively enrolled. Alemtuzumab was administered subcutaneously and concentrations were measured. These results are shown in FIG. 1. Here, all patients received population PK model-informed reduced alemtuzumab dosing of 0.5-0.6 mg/kg administered subcutaneously and divided over three days starting on day −14. Patients less than 15 kg were given 0.6 mg/kg divided equally over three days (0.2 mg/kg/dose). Patients greater than 15 kg were given a 3 mg test dose (maximum initial dose per manufacturer guidelines) on day −14 followed by approximately 0.23 mg/kg/dose on days −13 and −12 for a total dose of 0.5-0.6 mg/kg.

Blood samples were drawn for alemtuzumab concentrations before the first dose of alemtuzumab, 15 minutes prior to the third dose, 8 and 24 hours following the third dose, and then daily through day 0. Alemtuzumab concentrations were measured as previously described.^{17, 23} PK modeling was performed on day −5 to predict day 0 concentrations. If alemtuzumab concentrations were predicted to fall below 0.15 μg/mL, simulations were performed to identify the individual “top-up” dose needed to target the day 0 concentration to within the target window, and a top-up dose of subcutaneous alemtuzumab was given on day −3.

One patient received a top-up dose of alemtuzumab on day −1 after repeat modeling with additional time points, as the first modeling predicted day 0 concentration to be right at the lower limit of the therapeutic window. Conditioning also included fludarabine 150 mg/m² for patients ≥10 kg or 5 mg/kg for patients <10 kg (divided over days −8 to −4) and melphalan 140 mg/m² for patients ≥10 kg or 4.7 mg/kg for patients <10 kg (given on day −3). GVHD prophylaxis consisted of cyclosporine plus mycophenolate mofetil or methylprednisolone except in 5 patients who received CD34+ selected grafts, where T-cell depletion was the only GVHD prophylaxis. Patients received antimicrobial prophylaxis, immunoglobulin replacement, and supportive care per standard practice.

### Pharmacokinetic Analysis

A population PK model was previously developed for subcutaneous alemtuzumab administration with nonlinear mixed effect modeling using NONMEM software (version 7.2, ICON, Ellicott City, Md.) as reported (Dong et al, manuscript submitted). The model was used to predict patient day 0 concentrations using alemtuzumab concentrations measured through day −6 as described above. Additional simulations were performed using a specialized clinical pharmacology software MW/Pharm (Mediware, Prague, Czech Republic) (24) in patients who were predicted to have day 0 concentrations below the therapeutic target window to determine alemtuzumab top-up doses to be given on day −3 as described above. One patient was observed to have a precipitous decline in alemtuzumab concentrations after day −5. Leftover PK samples were used to measure anti-alemtuzumab antibodies as previously described,²⁵ and anti-alemtuzumab antibodies were detected at low concentrations.

### Clinical Outcomes

Neutrophil recovery was defined as the first of 3 consecutive days with a peripheral blood absolute neutrophil count >0.5×10⁹/L. Acute GVHD was diagnosed and graded according to consensus guidelines.²⁶ Peripheral blood cytomegalovirus (CMV), Epstein-Barr virus (EBV) and adenovirus PCRs were monitored once or twice weekly in all patients per institution standards. Whole blood chimerism was evaluated in the clinical laboratory via short tandem repeat analysis in the case of same sex donors and XY-fluorescent in-situ hybridization analysis in the case of opposite sex donors. Primary graft failure was defined as failure of initial engraftment of donor cells within the first 28 days post-HCT, and secondary graft failure was defined as loss of donor cells after initial engraftment. Mixed chimerism was defined as detection of <95% donor chimerism. CD4+ and CD8+ T cell, NK cell and CD19+ B cell counts were collected from clinically performed lymphocyte subset enumeration on day +100 (+/−14 days). The probability of event-free survival was calculated using the Kaplan-Meier method. An event was defined as primary graft failure, secondary graft failure or receipt of a second transplant (death was not included as an event as there were no deaths in the cohort).

### Statistical Analysis

Binary and categorical variables were summarized by frequency (%), and continuous variables were summarized by median with range. The Mann-Whitney test was performed for single comparisons of continuous variables between independent groups.

### Results

### Patients

Twelve patients were prospectively enrolled in this pilot study. One patient (Patient 8) with severe aplastic anemia developed recurrence of cholecystitis shortly after completing the three-day alemtuzumab dosing, and HCT was delayed. This patient was included in alemtuzumab PK analyses but excluded from analyses of clinical outcomes. Patient and transplant characteristics of the remaining 11 patients with a median age of 10 years (range 1-21 years) are shown below in Table 1.

TABLE 1
Patient and Transplant Characteristics

| Characteristic | N = 11 | |
|---|---|---|
| Age (yr), median (range) | 10 | (1-21) |
| Diagnosis, n (%) | | |
| Bone marrow failure (BMF) | 5 | (45.5%) |
| Hemophagocytic lymphohistiocytosis (HLH) | 3 | (27.3%) |
| Glanzmann's thrombasthenia | 1 | (9.1%) |
| Erythropoietic protoporphyria | 1 | (9.1%) |
| Artemis (DCLRE1C)-Deficient Leaky SCID | 1 | (9.1%) |
| Donor, n (%) | | |
| 10/10-HLA matched related | 2 | (18.2%) |
| 10/10 HLA-matched unrelated | 6 | (54.5%) |
| 9/10 HLA-matched unrelated | 2 | (18.2%) |
| 8/10 HLA-matched related | 1 | (9.1%) |
| Graft, n (%) | | |
| Bone marrow | 5 | (45.4%) |
| CD34+ selected peripheral blood stem cells | 5 | (45.4%) |
| Unmanipulated peripheral blood stem cells | 1 | (9.1%) |
| Total nucleated cell dose (×10\hat{ }8/kg), median (range) | 6.1 | (2.7-36.3) |
| CD34+ cell dose (×10\hat{ }6/kg), median (range) | 7.1 | (1.9-32.0) |
| Graft-versus-host disease prophylaxis, n (%) | | |
| Cyclosporine + mycophenolate mofetil | 3 | (27.3%) |
| Cyclosporine + methylprednisolone | 3 | (27.3%) |
| None * | 5 | (45.5%) |

BMF = 3 Severe Aplastic Anemia, 1 Kostmann Syndrome, 1 Paroxysmal Nocturnal Hemoglobinuria; HLH = 2 UNC13D Deficiency, 1 STXBP2 Deficiency; SCID = Severe Combined Immunodeficiency;
* Patients who received CD34+ selected grafts/T cell depletion did not receive additional graft-versus-host disease prophylaxis.

### Observed Day 0 Alemtuzumab Concentrations

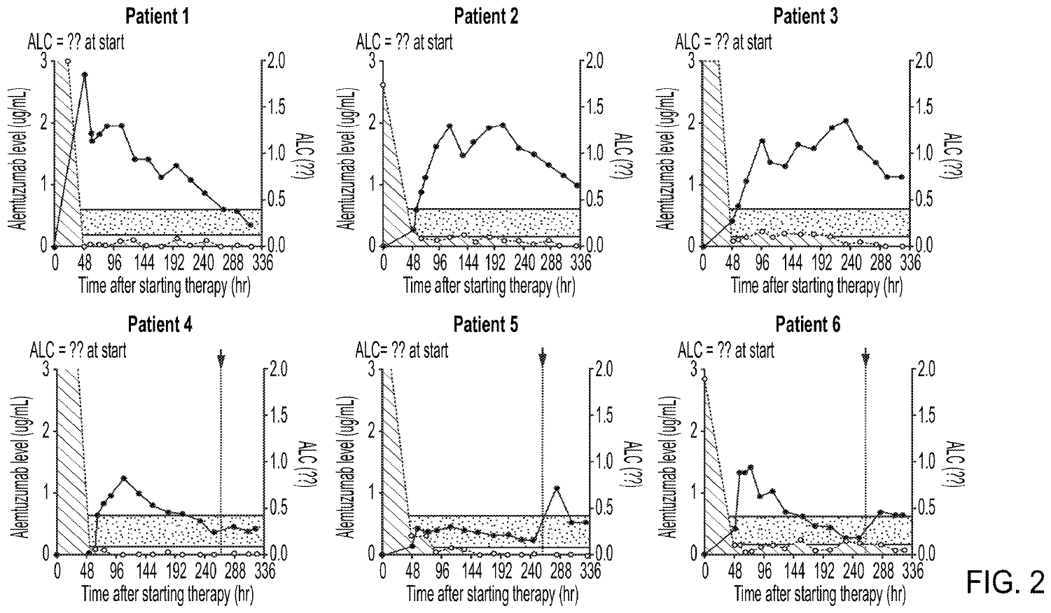

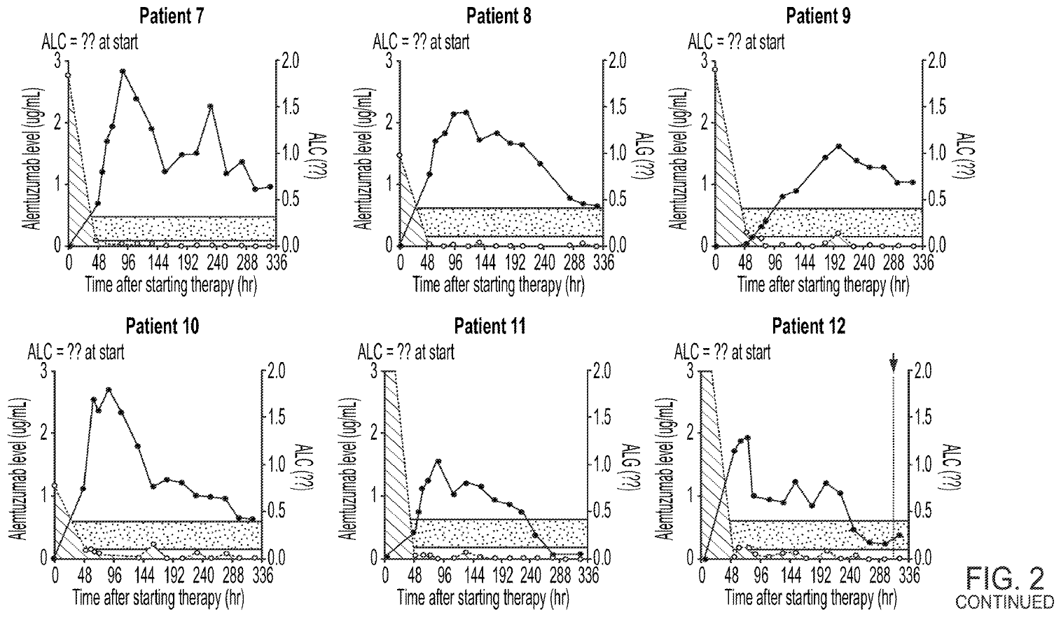

Patient absolute lymphocyte counts and observed alemtuzumab concentrations over time are shown for all 12 enrolled patients, as shown in FIG. 2. Lymphocytes were rapidly depleted in all patients, and depletion of lymphocytes coincided with an increase in alemtuzumab concentration. Overall, the median day 0 alemtuzumab concentration was 0.6 μg/mL (range 0.05-1.1). Two patients had day 0 alemtuzumab concentrations within the target window of 0.15-0.6 μg/mL without the need for top-up alemtuzumab doses (patients 1 and 10). Three patients (patients 4, 5 and 6) received a top-up dose of alemtuzumab on day −3 based on PK modeling predicting a day 0 concentration <0.15 μg/mL, and all three patients achieved alemtuzumab concentrations within the therapeutic window on day 0. One patient (patient 12) received a top-up dose on day −1 after repeat modeling with additional time points, as the first modeling predicted day 0 concentration to be right at the lower limit of the therapeutic window. This patient also achieved a day 0 alemtuzumab concentration within the target window. Five patients had day 0 concentrations above the target window (patients 2, 3, 7, 8, and 9) with a median day 0 alemtuzumab concentration of 1.04 μg/mL (range 0.66-1.12) (none of these patients received a top-up dose). Finally, one patient's (patient 11) alemtuzumab concentration was predicted to fall within the target window on day 0. However, alemtuzumab concentrations fell precipitously after day −5, and their day 0 alemtuzumab concentration was below the therapeutic window. Anti-alemtuzumab antibodies were detected at low concentrations and may explain the unanticipated rapid decline (data not shown).

### Influence of Absolute Lymphocyte Counts

We previously reported a negative correlation between pre-transplant absolute lymphocyte counts and day 0 alemtuzumab concentrations.¹⁶ Similarly, patients who required top-up doses of alemtuzumab in this study had, on average, higher absolute lymphocyte counts prior to alemtuzumab than those patients who fell within or above the therapeutic target window of alemtuzumab. This observation, however, did not reach statistical significance (median of 2.86×10\hat{ }3 cells/μL (range 1.86-6.52) versus 1.33×10\hat{ }3 cells/μL (range 0.77-3.64), p-value=0.16).

### Accuracy of Model Predictions

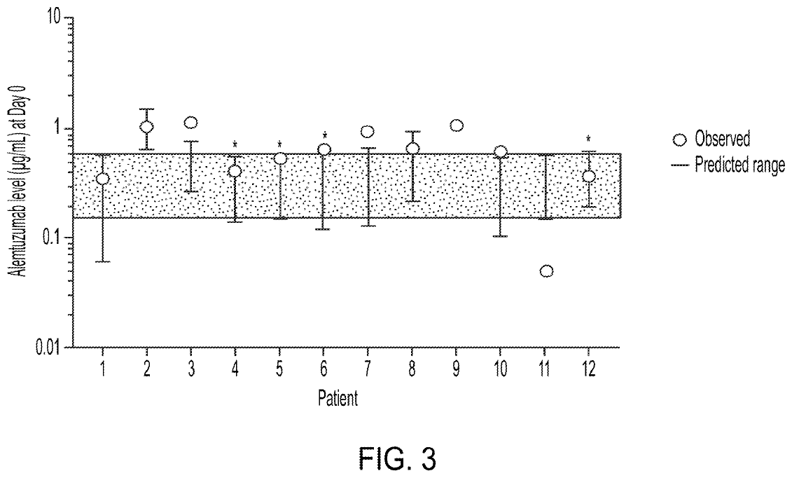

The observed and model-predicted day 0 alemtuzumab concentrations are shown in FIG. 3 to illustrate the accuracy of PK modeling predictions. A prediction was not made for one patient (patient 9) whose alemtuzumab concentrations were still increasing on day −6. Overall, six (55%) of 11 patients had measured day 0 alemtuzumab concentrations within the model predicted ranges. Four (36%) patients had concentrations above the predicted ranges. The patient who developed low concentration anti-alemtuzumab antibodies had a day 0 concentration below the model-predicted range.

Comparison to the Previous Cohort of Patients Treated with Standard Intermediate Alemtuzumab Dosing

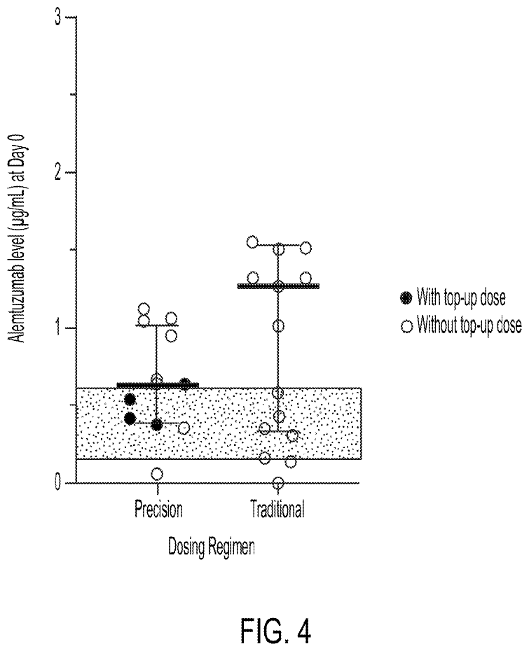

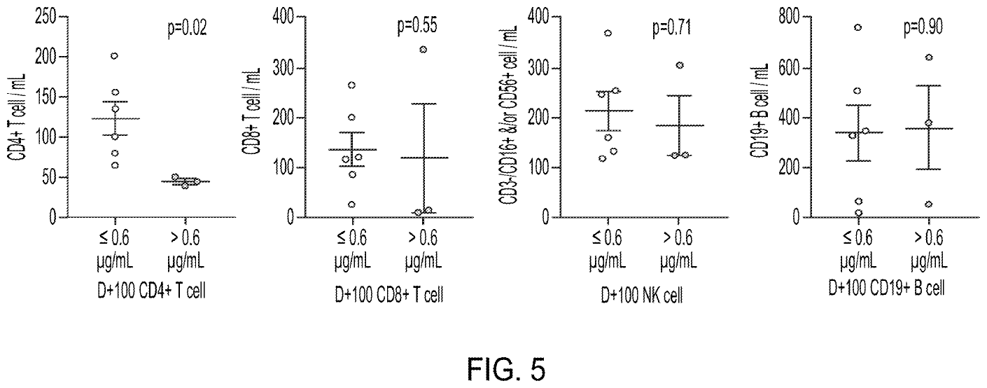

FIG. 4 shows day 0 alemtuzumab concentrations in this cohort compared to day 0 alemtuzumab concentrations in our previously published cohort of 17 patients with primary immunodeficiency who received standard intermediate alemtuzumab dosing (1 mg/kg alemtuzumab administered subcutaneously and divided over 5 days starting on day −14).¹⁷ Fifty percent of patients who received precision alemtuzumab dosing in this current study had day 0 concentrations within the target therapeutic window compared to only 24% of patients who received traditional intermediate alemtuzumab dosing. Additionally, the median day 0 alemtuzumab concentration was lower with a narrower range (median 0.6 μg/mL; range 0.05-1.1) in this study compared to the previous study (median 1.3 μg/mL; range 0-2.6) (p=0.11).

### Clinical Outcomes

Ten (91%) of the 11 patients eligible for assessment of clinical outcomes achieved primary neutrophil engraftment on median of day +12 (range 10-15 days). One (9%) patient with a day 0 alemtuzumab concentration of 0.53 μg/mL had primary graft failure followed by autologous recovery and was successfully re-transplanted 8 months later. Of note, this patient was found to have donor-specific anti-HLA antibodies that may have contributed to graft failure.

Six of the 10 patients with primary engraftment had complete (>95%) whole blood donor chimerism at day +100, and donor chimerism remained high (>85%) in all 6 patients on last check at 8-20 months post-HCT. Four of the 10 patients with primary engraftment had mixed chimerism with a median donor chimerism of 57.2% (range 3.4-78.7) at day +100. One of these patients with aplastic anemia developed secondary graft failure on day +63 and received a CD34+ selected stem cell boost without response (2.2% donor chimerism pre-boost versus 3.4% donor chimerism post-boost at day +100). The patient ultimately had autologous recovery and did not receive a second HCT. He remains clinically stable with stable blood counts on eltrombopag 14 months post-HCT. The second patient received a CD34+ selected stem cell boost on day +88 for mixed chimerism but continued to have low donor chimerism (29.6% donor at day +100). This patient ultimately received a second allogeneic HCT 14 months later and successfully engrafted. The remaining two patients with mixed chimerism (patients 1 and 6) had day +100 whole blood donor chimerism of 78.6% and 70%, respectively, and 1-year whole blood donor chimerism of 37% and 100%, respectively. The development of mixed chimerism was not associated with higher day 0 alemtuzumab concentrations. The median day 0 alemtuzumab concentration was 0.78 μg/mL (range 0.05-1.06 μg/mL) in those with full donor chimerism versus 0.52 μg/mL (range 0.35-1.2 μg/mL) (p>0.99) in those with mixed chimerism.

One patient developed Grade III acute gastrointestinal GVHD. Cyclosporine had been abruptly stopped in this patient due to concern for developing posterior reversible encephalopathy syndrome shortly prior to the onset of GVHD. The day 0 alemtuzumab concentration in this patient was well above the lytic concentration at 1.04 μg/mL.

FIG. 5 shows patients with day 0 alemtuzumab concentrations within or below the therapeutic target window had significantly higher day +100 absolute CD4+ T cell counts than those with day 0 alemtuzumab concentrations above the target window (median of 119 cells/μL [range 66-202] versus 46 cells/μL [range 39-52], p=0.02). There was no significant difference in CD8+ T cell, NK cell or CD19+ B cell counts.

Four patients developed viral reactivation: one developed CMV reactivation, 3 developed EBV reactivation, and 3 developed adenovirus reactivation. The patient with CMV reactivation and one of the patients with EBV reactivation received donor-derived virus-specific T cells. All four patients recovered from their viral reactivations. The development of viral reactivation was not associated with day 0 alemtuzumab concentrations in this cohort.

Ultimately, all 11 patients are alive and well (100% overall survival) at a median of 14 months (range 10-20) follow-up. Estimated one-year event-free survival was 71.6% (95% CI 44.2-99.0%).

### Discussion

This is the first prospective study of model-informed reduced dose alemtuzumab with a target concentration intervention strategy as part of RIC HCT. Our strategy led to twice the number of patients achieving alemtuzumab concentrations within the target concentration window of 0.15-0.6 μg/mL compared to our previous report of patients who received standard intermediate alemtuzumab dosing. Importantly, all four patients who received top-up doses had day 0 alemtuzumab concentrations within the target window, indicating that our target concentration intervention strategy is able to accurately course correct patients who would otherwise have non-lytic concentrations at day 0. Excluding the patient who developed anti-alemtuzumab antibodies, there were no patients with observed sub-therapeutic day 0 alemtuzumab concentrations.

Five patients had day 0 alemtuzumab concentrations above the target window and therefore increased potential for mixed chimerism and delayed immune reconstitution despite using a reduced alemtuzumab dosing schedule. These results suggest the need for further initial dose de-escalation to increase the proportion of patients who are able to achieve the target window with the target concentration intervention strategy.

With regard to clinical outcomes, we effectively minimized the risk of GVHD by avoiding non-lytic alemtuzumab concentrations on day 0. Only one patient developed acute GVHD, which was associated with abrupt cessation of GVHD prophylaxis. However, a high proportion of patients (40% of engrafted patients) developed mixed chimerism, and two of the four patients who developed mixed chimerism needed further cellular intervention. This is similar to the rate of mixed chimerism previously reported with standard intermediate alemtuzumab dosing used with fludarabine and melphalan RIC; in our prior study, 42% of patients with day 0 alemtuzumab concentrations of 0.16-4.35 μg/mL developed mixed chimerism. Interestingly, the development of mixed chimerism in the current cohort was not associated with high day 0 alemtuzumab concentrations. The lack of association between day 0 alemtuzumab concentration and development of mixed chimerism may be reflective of the small cohort size or the relatively narrow day 0 alemtuzumab concentration window. It may also indicate that even with optimization of alemtuzumab concentrations, there is a need to incorporate further myelosuppression to alemtuzumab, fludarabine, and melphalan RIC regimens. One prior study has in fact demonstrated high rates of complete donor chimerism with the addition of hydroxyurea and thiotepa. We did again observe that patients with day 0 alemtuzumab concentrations within or below the ideal therapeutic window have significantly higher CD4+ T cell counts at day +100, similar to our previous observations.

Our approach has some limitations. Firstly, the high number of blood samples used for each patient to perform PK modeling and predictions is not realistic in the wider clinical setting. As such, it will be essential to identify the key samples necessary for accurate PK modeling to develop an optimal sampling technique going forward. Additionally, with future studies including larger numbers of patients, it would be desirable to identify parameters in addition to weight that correlate with alemtuzumab bioavailability and clearance to allow for further individualized dosing and decrease reliance on top up doses. Finally, in the prior study that identified an ideal therapeutic window for day 0 alemtuzumab concentrations of 0.15-0.6 μg/mL, the vast majority (96%) of patients received bone marrow grafts. Receipt of other types of grafts with differing lymphocyte content may affect alemtuzumab clearance. As such, it is possible that the ideal therapeutic window may differ by graft source, and this requires further investigation.

This study demonstrates that model-informed reduced dosing of alemtuzumab with a target concentration intervention strategy can successfully achieve day 0 concentrations the ideal therapeutic window of 0.15-0.6 μg/mL in approximately half of patients. Subsequent studies are needed with further initial alemtuzumab dose de-escalation and optimization of the regimen to achieve target attainment in all patients. Adjustments to the intensity of the chemotherapeutics may also be needed to further decrease the incidence of mixed chimerism. These modifications are likely to further improve outcomes for pediatric patients with non-malignant diseases undergoing HCT by preventing GVHD while maximizing donor chimerism and early immune recovery.

### Example 2—Model-Informed Precision Dosing for Alemtuzumab in Pediatric and Young Adult Patients Undergoing Allogeneic HCT

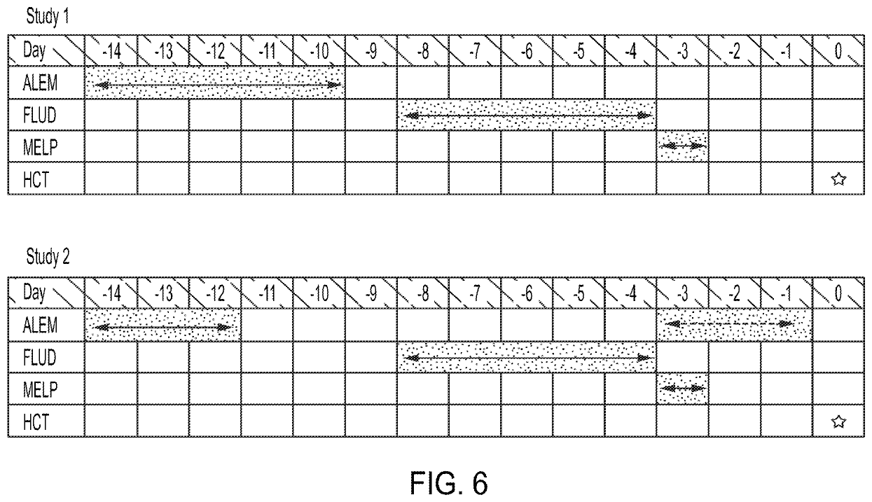

Data were collected from 29 pediatric and young adult patients with non-malignant disorders undergoing HCT enrolled in two studies. Alemtuzumab was administered subcutaneously with fludarabine and melphalan under the designated RIC regimen prior to HCT, as shown in FIG. 6.

Alemtuzumab dosing for Study 1 and Study 2 was as follows:

Study 1 (n=17): This study was previously reported by our team [4]. Briefly, alemtuzumab was given as a total dose of 1 mg/kg on Days −14 to −10 (Days 14 to 10 before the transplant date). For patients with body weight <15 kg, the total dose was divided equally over 5 days. For patients with body weight ≥15 kg, the first dose was limited to 3 mg (the manufacturer's recommended maximum initial dose) and the remainder of the 1 mg/kg total dose was divided over the remaining 4 days. Blood samples were drawn for PK measurement at predose, and 0.5, 2, 4, 6, 8 hours after the first two doses, and 0.5, 8 hours after the 3rd and 4th doses. Daily concentration measurements started from day 5 until graft infusion.

Study 2 (n=12): This study was approved by the institutional review board and is described and registered at Clinicaltrials.gov (NCT03302754). Informed consent was obtained for all patients. For patients with body weight <15 kg, alemtuzumab was given as a total dose of 0.6 mg/kg on Days −14 to −12 (0.2 mg/kg/dose). For patients with body weight ≥15 kg, the first dose was limited to 3 mg and the following doses were approximately 0.23 mg/kg/dose on days −13 and −12 (to equal a total dose of approximately 0.5-0.6 mg/kg). Based on a preliminary PK analysis, patient specific PK parameters were derived based on concentration measurements. These individual PK parameters were next utilized to predict the Day 0 concentrations. For patients who were projected to clear alemtuzumab by Day 0 to less than 0.15 μg/mL, different top-up dose levels were simulated, and the dosage that would lead to achieve the exposure target was selected as the top up dose, given either on Day −3 or on Day −1. The plasma samples were collected 15 minutes prior to the third dose, 8 and 24 hours following the third dose, and then daily through day 0.

Alemtuzumab concentrations in plasma were quantified by a validated flow cytometric assay according to the method previously reported.

Population PK-PD analysis was performed by nonlinear mixed effect modeling using NONMEM (ICON, Ellicott City, Md., USA). Perl speaks NONMEM (PsN) version 3.6.2 [10] and Pirana version 2.7.1 (Certara USA, Inc., Princeton, N.J., USA) were used as the interface. NONMEM 7 version 7.4 with the Stochastic Approximation Expectation-Maximization (SAEM) estimation algorithm, followed by important sampling (IMP) method was employed to estimate the typical population parameters, random effect of inter-individual variability, and residual variability simultaneously. The IMP's expectation step only (EONLY=1) was implemented in the evaluation of the marginal likelihood objective function but retaining the fixed effects values at the final answer of the SAEM method The inter-individual variability was assessed using the following model (Equation 1):

Pᵢ=Pₚₒₚ×exp(ηᵢ) (1)

where Pᵢ is the estimated parameter value for individual i; Pₚₒₚ is the typical population value of the PK or PD parameters such as clearance and volume of distribution; Ty is an inter-individual random effect for individual i. The residual unexplained variability was described by a proportional error model (Equation 2) or an exponential model (Equation 3).

Y_{i,j}=C_{pred,i,j}×(1+εₚᵣₒₚ) (2)

Y_{i,j}=C_{pred,i,j}×exp(εₑₓₚ) (3)

where Y_{i,j} is the observed concentration, C_{pred,I,j} is the predicted concentration for individual I, and ε is a residual variability.

During the mode model development process, a one- or a two-compartment model with 1^{st}-order linear kinetics disposition or nonlinear Michaelis-Menten disposition were evaluated. As subcutaneous alemtuzumab absorption has not been quantitatively described, one of our modeling foci was to identify the best absorption model. The following absorption model structures were evaluated: 1) a first order absorption with or without a lag time; 2) a sequential linked or unlinked zero- and first-order absorption, and 3) a parallel zero- and first-order absorption processes.

For the population PK-PD modeling, both sequential and simultaneous approaches were initially evaluated. The sequential approach eventually was used as it provided non-inferior performance than simultaneous fitting and was much more efficient in computational time (minutes versus hours in running time). PK-PD model structures including direct and indirect linear, inhibitory Eₘₐₓ, and sigmoidal Eₘₐₓ models were evaluated. ALC counts that were below the limit of quantification (i.e. <0.01 k/μl) were excluded from the analysis as these measurements did not reflect quantitative PK-PD correlation changes.

### Covariate Analysis

Demographic and laboratory characteristics of the patients including body weight, age, baseline ALC, white blood count (WBC) and albumin levels at start of alemtuzumab treatment, along with daily measured ALC and WBC during the treatment were evaluated as potential covariates using the stepwise selection method. The change in the objective function value (OFV) between two nested models was assumed to follow the χ2 distribution, and forward inclusion and backward elimination with a significance level of <0.05 (−3.84 points in OFV) and <0.01 (−6.64 points in OFV) were used, respectively. In accordance with allometry theory, body weight was found to be significantly correlated to alemtuzumab clearance (CL) and volume of distribution (V) at initially evaluation. We therefore used allometrically scaled body weight to account for differences in body 183 size as follows (Equation 4):

$${P}_{i} = {P}_{p o p} \times {( \frac{B {W}_{i}}{B {W}_{standard}} )}^{P o w e r} \qquad ( 4 )$$

where BWᵢ is body weight for individual i, BW_{standard} is 70 kg, and power is the coefficient set at 0.75 for CL and 1 for V. Other potential covariates were tested as formulated to a linear or power function as illustrated using ALC as an example (Equations 5 and 6):

$${P}_{i} = {P}_{p o p} \times ( 1 + ( \frac{A L {C}_{i}}{A L {C}_{m e d i a n}} ) \times l ) \qquad ( 5 )$$

$${P}_{i} = {P}_{p o p} \times {( \frac{A L {C}_{i}}{A L {C}_{median}} )}^{k} \qquad ( 6 )$$

where Pᵢ and ALCᵢ are the parameter and ALC at predose for individual i, ALC_{median} is the standardized value for ALC at predose. The l and k represent the slope and power factor of the relationship, respectively (i.e., Equations 5 and 6 for linear and power relationship equations, respectively).

### Model Evaluation

The following criteria were considered for model selection: successful convergence, objective function value (or Akaike Information Criterion), precision of parameter estimates, and plausibility of parameter estimates. In addition, diagnostic goodness-of-fit plots and graphical assessments were performed using R version 3.6.2 and Xpose version 4.4.0. The following diagnostic plots were used to evaluate the models: observed value (DV) vs. population predicted value (PRED), DV vs. individual predicted value (IPRED), conditional weighted residuals (CWRES) vs. PRED and CWRES vs. time after dose to identify a bias corresponding to model mis-specification. A non-parametric bootstrap analysis was employed to evaluate the stability of the final model using Perl-speaks-NONMEM (PsN) version 3.5.3. The resampling was done 500 times, and 95% confidence intervals of parameter estimates from the bootstrap analysis were compared to the final model estimates. The prediction-corrected visual predictive check (pcVPC) was used for final evaluation. One thousand replicates of simulated datasets were generated using the final model and the distribution of simulated observations was compared with the actual observations.

### Monte Carlo Simulation

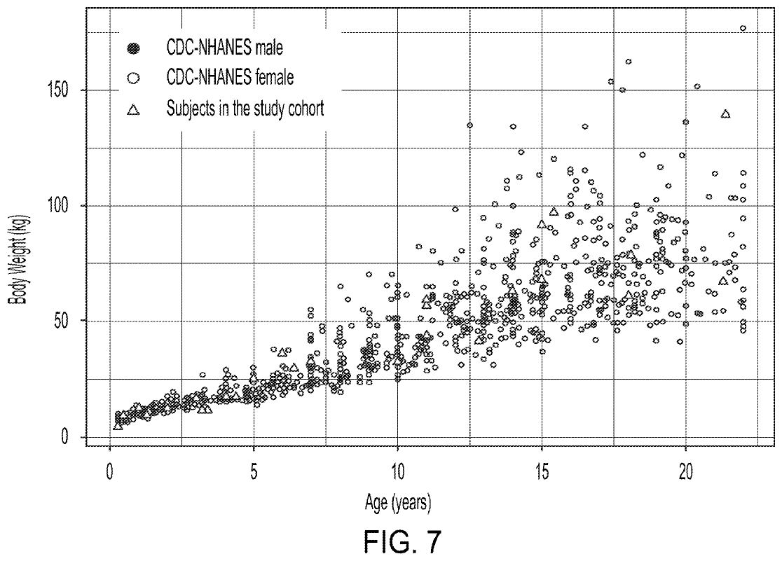

Monte Carlo simulation which considers both population average and inter-individual variability, was conducted to select an appropriate dose to achieve a peri-transplant alemtuzumab concentration of 0.15-0.6 μg/mL on the day of transplantation (Day 0). This target range is based on our previous findings on alemtuzumab efficacy and safety for the RIC regimen. A range of candidate cumulative doses based on three dose calculation methods were tested: 1) based on total body weight as mg per kg, as in the current dosing protocol; 2) based on total body weight using the allometric scaling principle: allometric dose=dose for a typical subject of 70 kg×(total body weight/70)0.75; 3) based on body surface area (BSA, mg per m²). The dosing scheme was designed as the total dose divided to 3 equal doses administered on Days −14 to −12 (14 days to 12 days before the transplant date). However, to ensure patient safety, the first dose on Day −14 could not exceed 3 mg. If the calculated first dose was above 3 mg, 3 mg was the set dose on Day −14 and the rest of the dose amount was equally divided to be administered over the remaining 2 days. In subjects with a predicted Day 0 concentration below 0.15 μg/mL, simulation-based individualized top-up dose was administered on Day −3. In the simulation analysis, we defined our target patient age range as 0.3-22 years to match the age range of the patient cohort. We used the patient characteristics from the US Centers for Disease Control and Prevention-National Health and Nutrition Examination Survey (CDCNHANES) database collected from 1999 to 2016, and only those records that have both weight and height (body length) information available are included in the dataset pool for sampling. This results in 41038 unique subjects. A total of 1,000 patients were randomly sampled from this data pool for each clinical trial simulation. The distribution of the weight-age cohorts from the sampled virtual subjects were plotted to assure similarity to the intended study treatment cohort, as shown in FIG. 7. The simulation dataset was constructed to estimate alemtuzumab concentration on a one-hour interval till the day of transplantation (14 days or 336 hours after the 1^{st} dose given). The percentage of subjects who had a projected day 0 concentration at 336 hours within the target exposure range (0.15-0.6 μg/mL) was summarized for each dosing regimen. All simulations to determine exposure levels of alemtuzumab associated with candidate dosing regimen were conducted using the R package mrgsolve.

### Statistical Analysis

Student t test was used for the comparison of patient characteristics between top-up and no top-up dose groups. Linear regression analysis was run to understand the correlation between alemtuzumab concentration on Day 0 and patient characteristics. A P value of <0.05 was considered significant.

### Results

### Population PK Modeling

The median age of the study subjects was 6.4 years (range: 0.28-21.4 years) and the median body weight was 32.0 kg (4.3-139 kg). Other patient demographics are summarized below Table 2.

TABLE 2
Demographic Summary

| | Age | BW | ALC | WBC | |
|---|---|---|---|---|---|
| | (Year) | (kg) | (k/μL)⁾ | (k/μL) | Diagnosis |
| Study 1 | N = 17 | | | | HLH (n = 10), CGD |
| Median | 7.0 | 32.2 | 1.03 | 7.1 | (n = 2), IPEX |
| Min | 0.5 | 8.92 | 0.06 | 0.4 | (n = 2), aplastic |
| Max | 18.0 | 91.5 | 6.0 | 12.5 | anemia (n = 1), CID |
| | | | | | (n = 1), SCID (n = 1) |
| Study 2 | N = 12 | | | | HLH (n = 3), SAA |
| Median | 5.4 | 23.3 | 2.28 | 3.9 | (n = 3), PNH (n = 1), |
| Min | 0.28 | 4.32 | 0.53 | 1.0 | Glanzmann's (n = 1), |
| Max | 21.4 | 139 | 6.52 | 9.1 | Kostmann's syndrome |
| | | | | | (n = 1), idiopathic |
| | | | | | aplastic anemia |
| | | | | | (n = 1), erythropoietic |
| | | | | | protoporphyria |
| | | | | | (n = 1), leaky SCID |
| | | | | | (n = 1) |
| Total | N = 29 | | | | |
| Median | 6.4 | 32.0 | 1.27 | 4.1 | |
| Min | 0.28 | 4.32 | 0.06 | 0.4 | |
| Max | 21.4 | 139 | 6.52 | 12.5 | |

HLH, hemophagocytic lymphohistiocytosis;
CGD, chronic granulomatous disease;
IPEX, immunodysregulation, polyendocrinopathy, enteropathy, X-linked;
CID, combined immune deficiency;
SCID, severe combined immune deficiency;
SAA, severe aplastic anemia

For PK modeling, a sequential zero- and first-order input model was found to best describe the alemtuzumab absorption profile after subcutaneous administration. A one-compartment model with first-order disposition adequately described alemtuzumab disposition. Allometrically scaled body weight was included in the model to account for body size related differences in volume of distribution and clearance with the theoretical values of 1 for volume and 0.75 for clearance. This inclusion resulted in a significant improvement in model fit (ΔOFV=−33.9, p<0.001). Of note, when the quantitative impact of weight on PK parameters was estimated as a power function as shown in Equation 4, the exponents were estimated as 0.67 for clearance and 1.17 for volume, respectively. Pre-dose absolute lymphocyte count (ALC) was not identified as a significant covariate of clearance (P>0.05), although a trend of negative correlation with alemtuzumab clearance was observed. No effects of age, gender, or albumin levels on alemtuzumab PK were observed. The inter-patient variability of clearance and volume of distribution were high (69.7% and 89.7%, respectively), and the duration of the zero order absorption process was highly variable, with a coefficient of variation (CV) of 225.6%. The population PK parameter estimates are summarized in Table 2. The population clearance estimate was 0.080 L/h/70 kg and the volume of distribution estimate was 17.4 L/70 kg.

Duration for zero order absorption is 6.77 hours and the 1st order absorption rate was 0.079 hour⁻¹. The final model is further illustrated as below.

During the zero-order period (first 6.77 hours after the SC injection), alemtuzumab was absorbed to a depot compartment with zero-order absorption:

Rate(mg/h)=Dose/6.77.

During the 1^{st} order period (after 6.77 hours of the SC injection), alemtuzumab was further absorbed to the central compartment with the 1^{st} order kinetics (absorption constant Ka):

$$C ( t ) = Dose * C * \frac{K a}{K a - K e} * ( {e}^{- Ket} - {e}^{- Kat} )$$

wherein C(t) is the drug concentration at the given time t; Dose is the alemtuzumab dose; Ka is the absorption rate constant; and Ke is the elimination rate constant and equals CL/V.

Individual clearance (CL/F__{ind}) and volume of distribution (V/F__{ind}) in relation to the only covariate in the model body weight (WTᵢ) are presented as:

CL/F__{ind}(L/h/70 kg)=0.080*(WTᵢ/70)^{0.75}

V/F__{ind}(L/h/70 kg)=17.4*(WTᵢ/70).

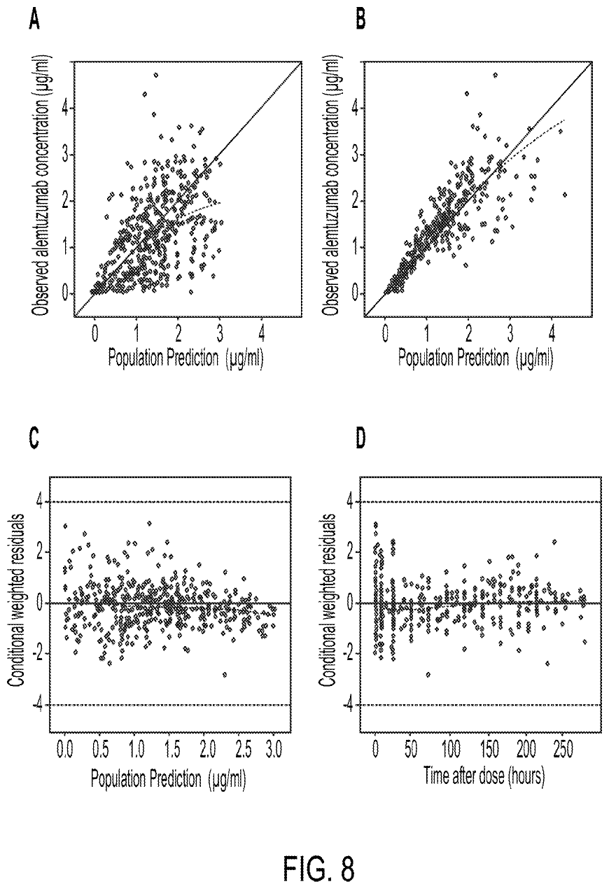

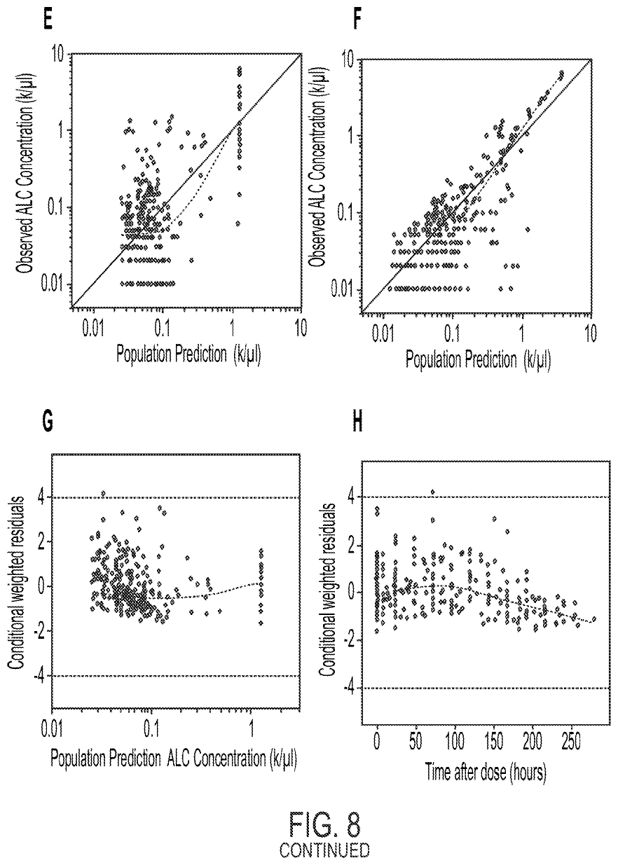

Goodness-of-fit plots indicated slight over predictions of high concentrations but showed an overall reasonable model fit, as shown in FIG. 8, particularly FIGS. 8A-8D.

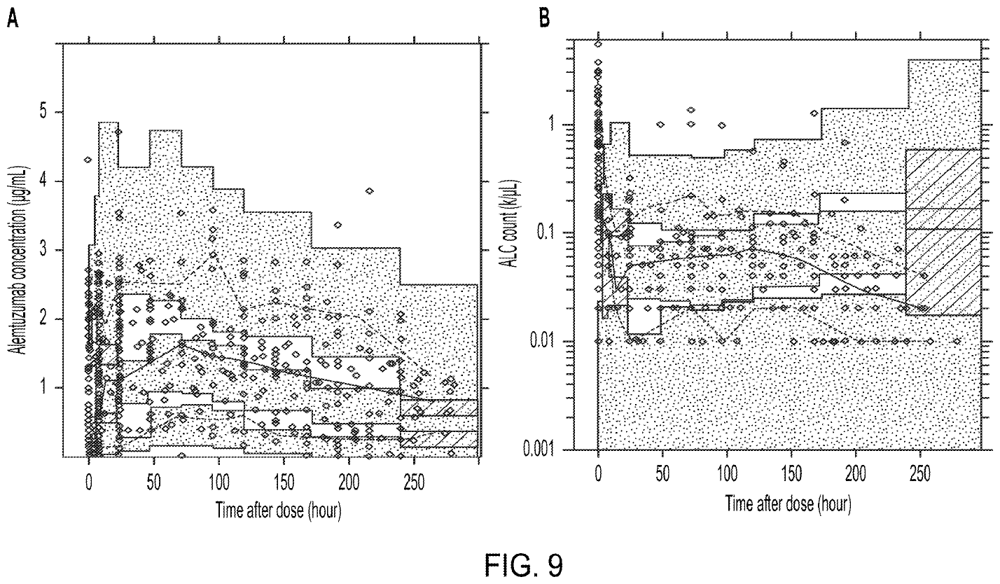

The non-parametric bootstrap analysis demonstrated stability of the model estimates on clearance and volume of distribution but high variability in absorption parameters and the results are summarized in Table 3. The simulated plasma concentrations by VPC analyses were in a reasonable agreement with the observed data. The 10th, 50th, and 90th percentile of predictions overlaid well 0.673 with their corresponding percentile of observations, as shown in FIG. 9, particularly FIG. 9A.

TABLE 3
Population Pharmacokinetic Parameter Estimates

| | | RSE | Bootstrap estimates | |
|---|---|---|---|---|
| Parameters (units) | Estimates | (%) | Median | 95% CI |

Fix-effect parameters

| | | | | |
|---|---|---|---|---|
| CL/F (L/h/70 kg) | 0.0795 | 14.5 | 0.0799 | 0.061-0.103 |
| V/F (L/70 kg) | 17.4 | 17.3 | 17.0 | 5.8-25.2 |
| kₐ(/h) | 0.079 | 26 | 0.094 | 0.032-1.774 |
| Duration for zero | 6.77 | 51 | 8.67 | 1.71-46.44 |
| order absorption (h) | | | | |
| Eₘₐₓ (k/μl) | 1.27 | 18.7 | 1.30 | 0.88-1.93 |
| EC₅₀ (μg/ml) | 0.062 | 27.3 | 0.059 | 0.032-0.108 |

Inter-patient variability (CV %)

| | | | | |
|---|---|---|---|---|
| IIV_{CL} | 67.7 | 17.3 | 67.8 | 42.0-93.2 |
| IIV_{V} | 62.5 | 15.4 | 83.6 | 0.09-227.1 |
| IIV_{Ka} | 101 | 20.2 | 109 | 0.05-353.2 |
| IIV_{DUR} | 183 | 19.1 | 193 | 66.6-322.4 |
| IIV_{Emax} | 85 | 22.3 | 77 | 22-106 |
| IIVEC₅₀ | 131 | 23.4 | 123 | 70-172 |
| Correlation of | −0.75 | 23.9 | −0.62 | (−1.21)-(−0.09) |
| Eₘₐₓ − EC₅₀ | | | | |

Residual variability

| | | | | |
|---|---|---|---|---|
| Prop.Err.Conc (CV %) | 39.8 | 3.8 | 39.3 | 29.7-48.2 |
| Add.Err.Effect (k/μl) | 0.01 | 41 | 0.00 | −0.07-0.09 |
| Prop.Err.Effect | 68.9 | 6.7 | 68.0 | −0.76-0.88 |
| (CV %) | | | | |

Clearance (CL/F) and volume of distribution (V/F), which are allometrically size-standardized with a power of 0.75 and 1.00, respectively;
Dur, Duration for zero order absorption;
RSE, relative standard error;
CI, confidence interval;
IIV, inter-individual variability;
CV, coefficient of variation;
Prop.Err.Conc, proportional part of the residual unexplained variability for alemtuzumab concentrations;
Add.Err.Effect, additive part of the residual unexplained variability for ALC count;
Prop.Err.Effect, proportional part of the residual unexplained variability for ALC count

### Population PK-PD Modeling

A direct inhibitory Eₘₐₓ model defined as shown below best characterized the PK-PD relationship between alemtuzumab concentration and ALC:

ALC=Eₘₐₓ*(1−Conc/(EC₅₀+Conc))

where ALC is the absolute lymphocyte count; Eₘₐₓ is the maximum inhibitory effect; EC₅₀ is the alemtuzumab concentration when half of the Eₘₐₓ is achieved; Conc represents the alemtuzumab concentration.

This model performed better in terms of overall fit and stability compared to a linear or a sigmoidal Eₘₐₓ model. We also evaluated an indirect response model as described in earlier studies, however this model structure was not supported by the data. The population Eₘₐₓ was estimated as 1.27 *103/μL and the EC₅₀ was 0.06 μg/mL. The observed inter-individual variability for Eₘₐₓ was 85% and was 131% for EC₅₀. Eₘₐₓ and EC₅₀ are correlated with a correlation coefficient of −0.7. Other final model parameters are presented in Table 3. Goodness-of-fit plots indicated over-prediction at the population level but was reasonable at the individual level, as shown in FIG. 8, particularly FIGS. 8E-811. Further model evaluation by bootstrap indicated that all parameter estimates were within less than 10% different from the population median levels. The VPC plot showed that the model predictions overall were adequate but trended higher than observations after 200 hours. As alemtuzumab concentrations decreased, ALC remained low, as shown FIG. 9, particularly FIG. 9B.

### Dose Optimization to Achieve Optimal Target Exposure on the Day of Transplantation (Day 0)

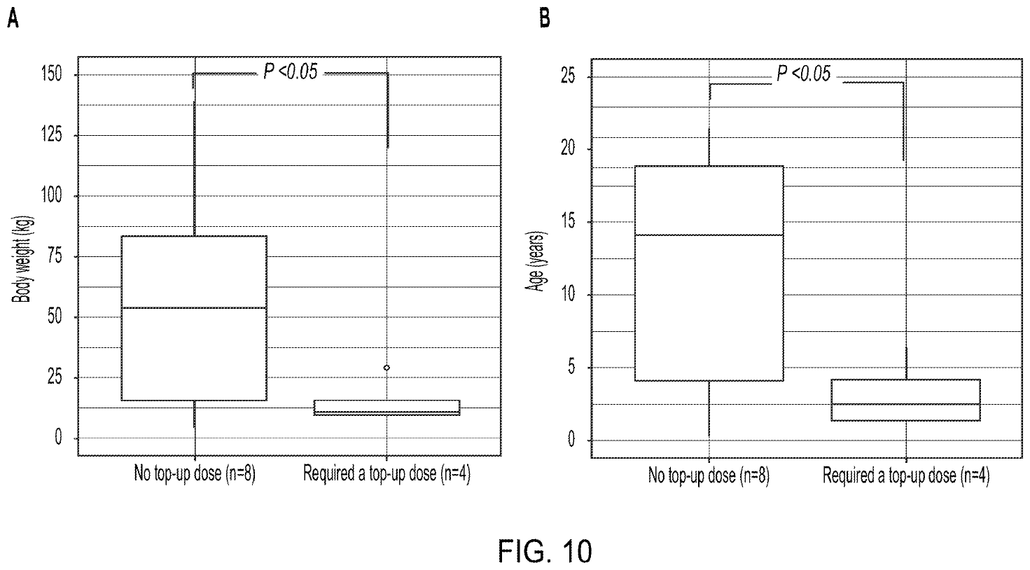

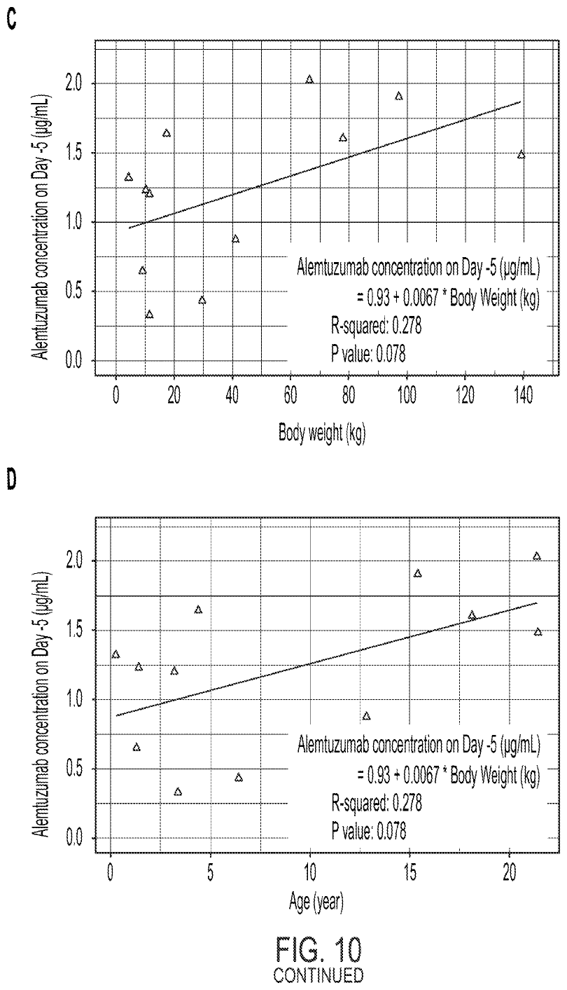

The current off-label use of alemtuzumab in pediatric patients is based on per kg dosing, which assumes a linear relationship between body weight and drug elimination/metabolism. In our pilot study (n=12), we observed that this per kg dosing results in an uneven alemtuzumab exposure across different age/weight spectra, as shown in FIG. 10. In patients who needed an additional top-up dose of alemtuzumab, body weight and age were significantly lower than in subjects who did not require a top up dose, as shown in FIG. 10, particularly FIGS. 10A-10B (p<0.05). In addition, alemtuzumab exposure on Day −5 was moderately correlated with age and body weight, with lower concentrations in the young/low weight patients and higher concentrations in the older/higher weight patients, as shown in FIG. 10, particularly FIGS. 10C-10D.

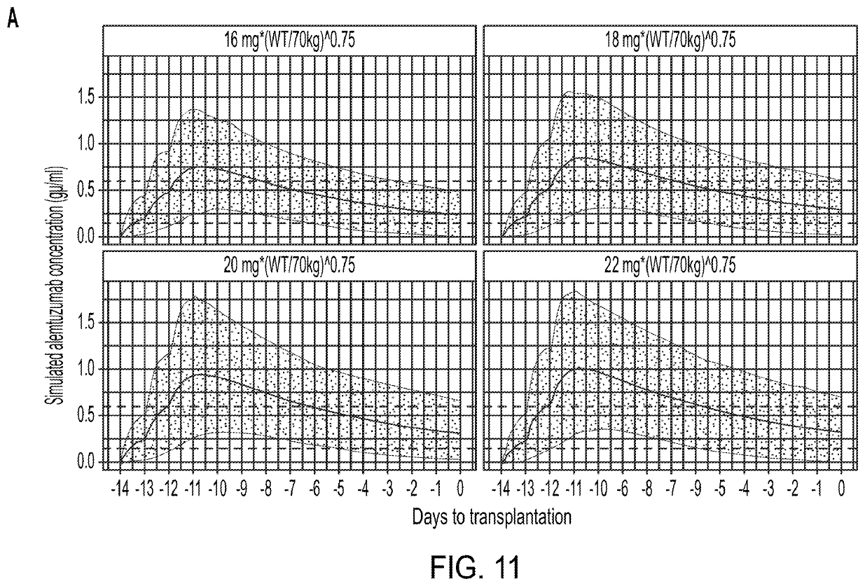

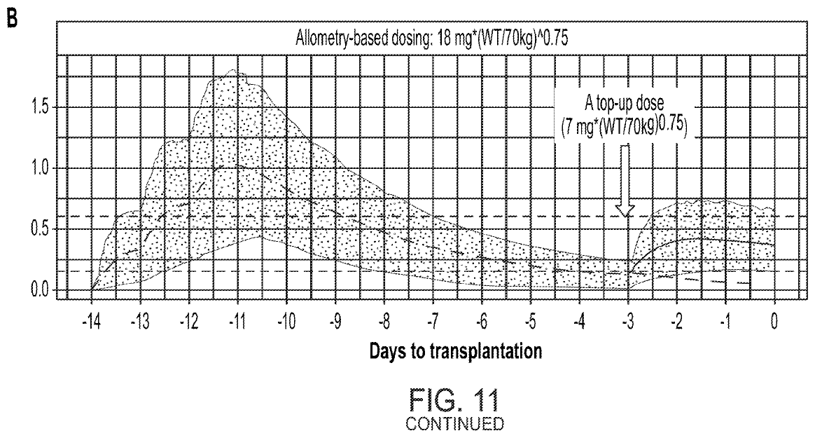

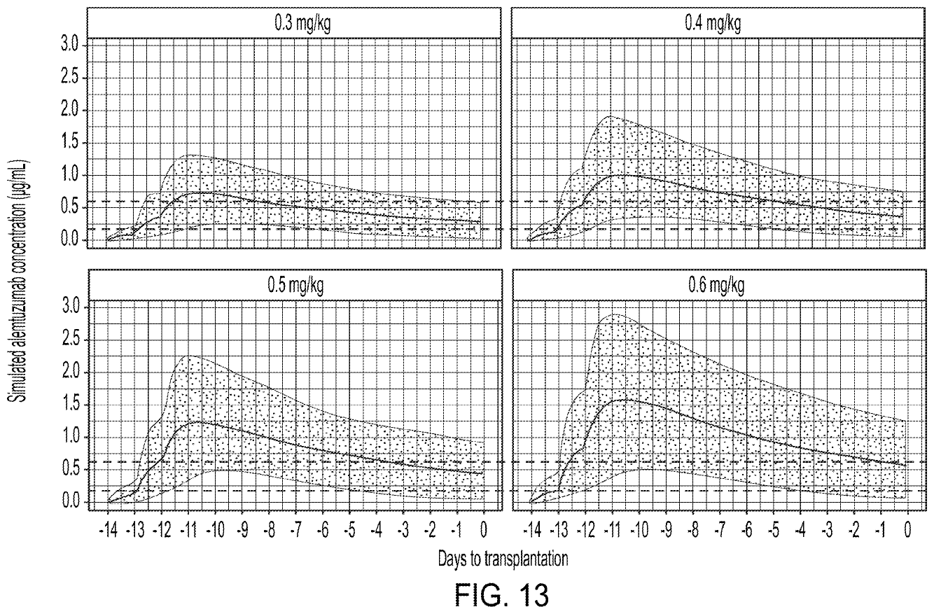

In light of this non-proportional correlation between alemtuzumab exposure with the per kg dosing and the allometric scaling applied in the population PK models, we conducted simulations to identify optimal starting dose with three dose calculation methods: 1) allometrically scaled dosing (individual dose=dose for a typical subject of 70 kg×(individual weight/70)0.75), 2) BSA-based dosing (mg per m²) and 3) body weight-based dosing (mg per kg). The PK profiles with candidate doses were simulated and the mean alemtuzumab concentrations with 10th-90th percentile of confidence intervals were evaluated in achieving the target concentrations of 0.15-0.6 μg/mL on Day 0. As shown in FIGS. 11, 12 and 13, alemtuzumab PKs show large inter-patient variability with broad prediction intervals with all three dosing methods.

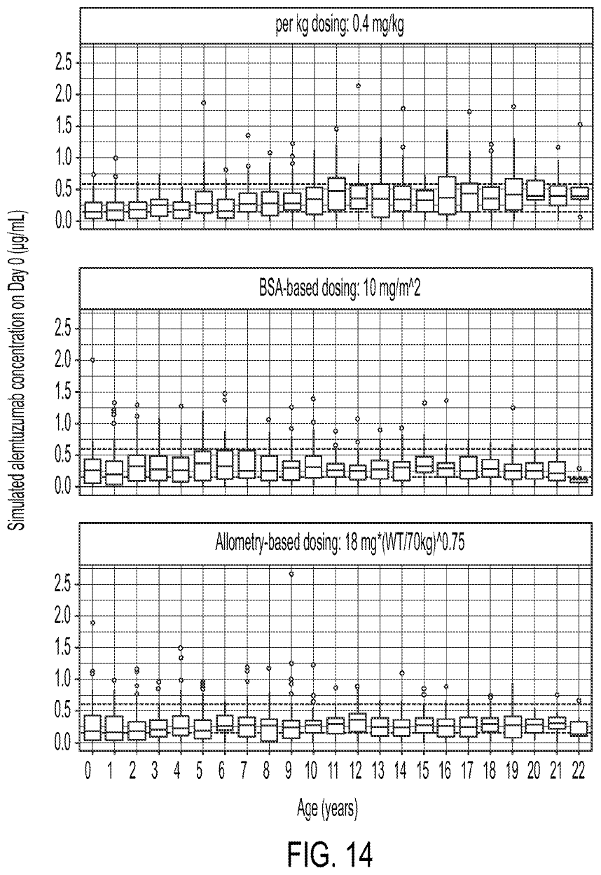

Per kg dosing would result in uneven alemtuzumab exposure across different age spectra, whereas BSA- or allometry-based dosing showed overall similar exposure levels in different age groups, as shown in FIG. 14. For allometry-based dosing, a dose level of 18 mg*(WT/70)0.75 would have the highest percentage of virtual patients (56.6%; i.e., 566 out of 1000 virtual patients) achieving the ideal therapeutic range of 0.15-0.6 μg/mL on Day 0, the results of which are summarized below in Table 4.

TABLE 4
Day 0 Target Achievement of Candidate Dosing Regimens

| | | | | |
|---|---|---|---|---|
| | Average Day 0 | Above | Within | Below |
| | concentration | 0.6 μg/ml | 0.15-0.6 | 0.15 μg/ml |
| Cumulative dose | (μg/ml) | (%) | μg/ml (%) | (%) |

Per KG dosing

| | | | | | |
|---|---|---|---|---|---|
| 0.3 | mg/kg | 0.26 | 7.6 | 54.0 | 38.4 |
| 0.4 | mg/kg | 0.33 | 15.7 | 52.6 | 31.7 |
| 0.5 | mg/kg | 0.45 | 28.9 | 45.2 | 25.9 |
| 0.6 | mg/kg | 0.56 | 38.5 | 38.6 | 22.9 |

Per BSA dosing

| | | | | | |
|---|---|---|---|---|---|
| 8 | mg/m² | 0.24 | 5.4 | 54.7 | 39.9 |
| 10 | mg/m² | 0.31 | 11.7 | 56.5 | 31.8 |
| 12 | mg/m² | 0.36 | 16.8 | 56.3 | 26.9 |
| 14 | mg/m² | 0.43 | 26.0 | 49.0 | 25.0 |

Allometry-based dosing

| | | | | |
|---|---|---|---|---|
| 16 * (WT/70)^{0.75} | 0.25 | 7.2 | 55.3 | 37.5 |
| 18 * (WT/70)^{0.75} | 0.28 | 9.1 | 56.6 | 34.3 |
| 20 * (WT/70)^{0.75} | 0.31 | 13.7 | 54.2 | 32.1 |
| 22 * (WT/70)^{0.75} | 0.34 | 16.4 | 53.5 | 30.1 |

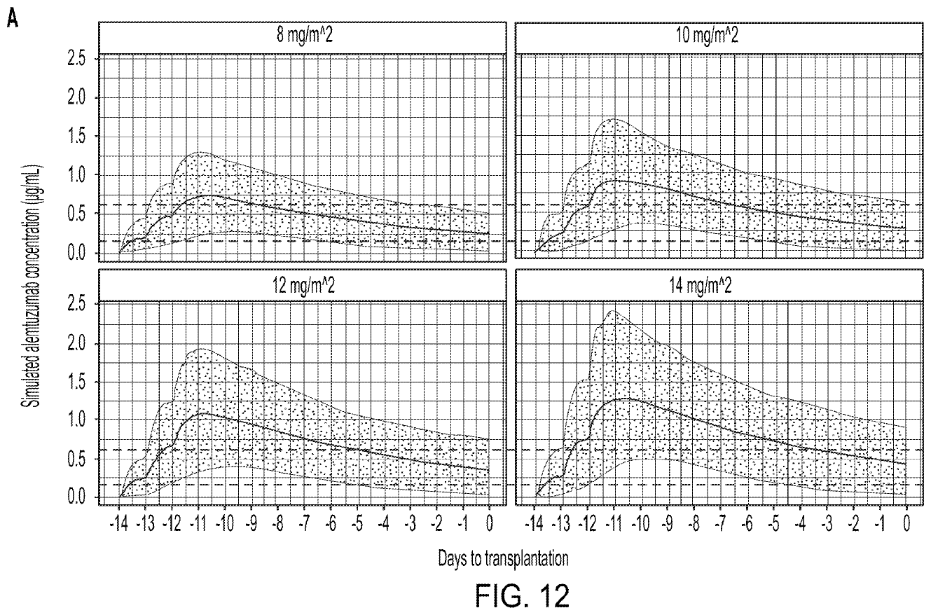

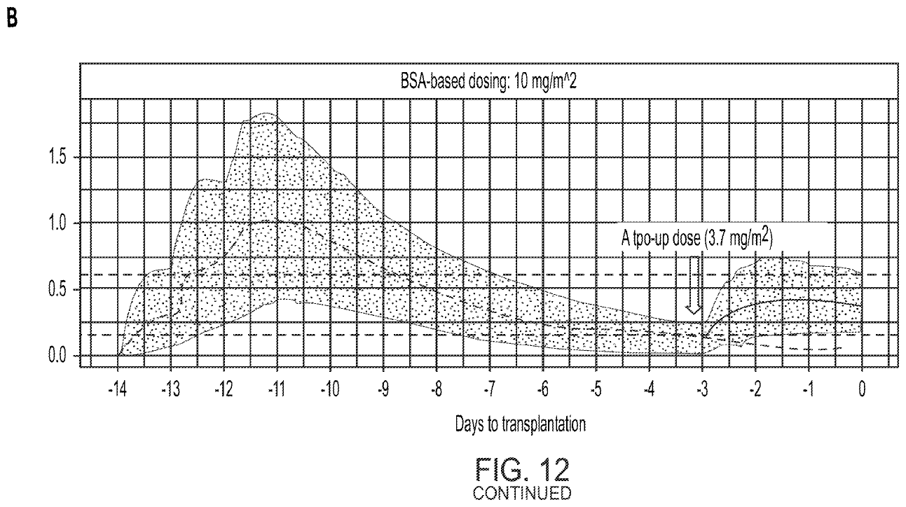

For BSA-based dosing, the dose level associated with the highest percentage of target attainment was 10 mg/m² (56.5%), see Table 4. Simulation analysis further indicated that in patients with a predicted alemtuzumab plasma concentration lower than the targeted 0.15 μg/mL, a top-up dose of 7 mg*(WT/70)0.75 for allometry-based dosing or 3.7 mg/m² for BSA-based dosing on Day −3 would bring the alemtuzumab concentration to the optimal range on Day 0 in 27.2% (i.e., 272 out of 1000 virtual patients) and 26.0% (i.e., 260 out of 1000 virtual patients) additional patients, respectively, as shown in FIGS. 11B and 12B.

### Discussion

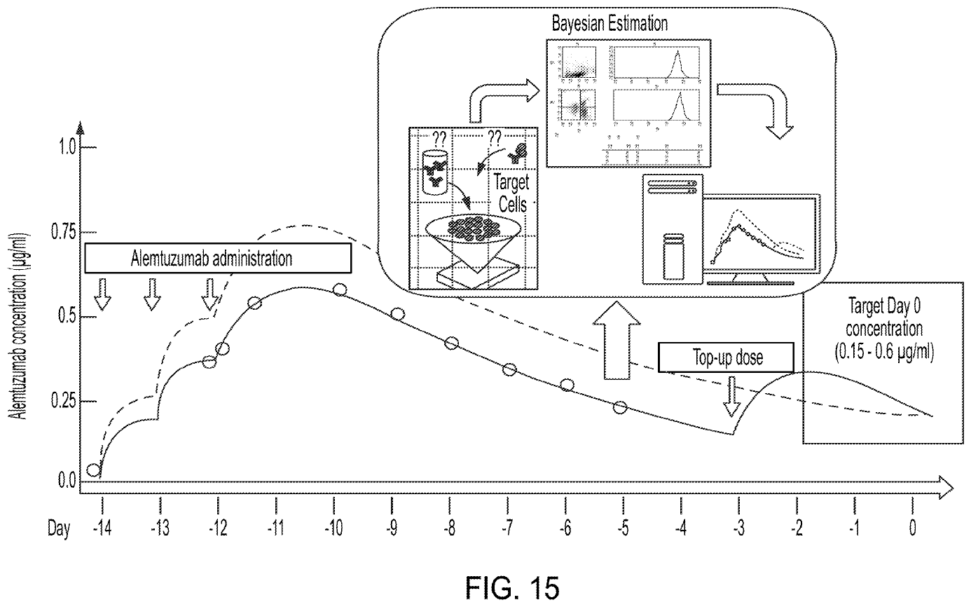

Our group previously reported the effect of alemtuzumab concentration on Day 0 (transplant day) on allogeneic HCT outcomes and identified an ideal therapeutic range of 0.15-0.6 μg/mL. A recent follow-up prospective pilot study however demonstrates that a reduced dosing schedule (cumulative dose of 0.5-0.6 mg/kg) is still too high in more than 40% of patients (manuscript submitted). Further dose de-escalation along with continued adaptive dose controls would be required to bring the majority of the patients into the proposed target range to prevent acute GVHD and to reduce the risk of mixed chimerism and delayed early post-HCT immune recovery. The current study characterized the population PK and PK-PD of alemtuzumab and conducted trial simulations with different dosing scenarios to identify the initial dose level for dose de-escalation. Based on the result, we propose consideration of a new dosing scheme for alemtuzumab which could be imbedded in a Bayesian algorithm for precision dosing as shown in FIG. 15 and tested in a future trial.

The current PK modeling practice advanced our preliminary analysis, and found that alemtuzumab PK could be best described by a one-compartment model with a zero- and first-order absorption, and first order elimination. The performance of this one-compartment model was non-inferior compared to a two-compartment model or a nonlinear Michaelis-Menten model according to the diagnostic plots and AIC values. However, others have described alemtuzumab PK by a 2-compartment or a Michaelis-Menten model. The disparity between studies could be due to different administration routes, dose levels, and PK sampling schemes. As a large antibody of 150-kDa, alemtuzumab is mostly confined in the plasma and interstitial space. After subcutaneous administration, the slow absorption to the central compartment (i.e. blood circulation) may limit alemtuzumab distribution to the extravascular space. In addition, our study subjects received relatively low doses (maximal single dose was 30 mg or 0.24 mg/kg) whereas in the adult study the maximal dose was 240 mg (approximately 3.3 mg/kg). This may explain why our data did not support a nonlinear Michaelis-Menten model. The estimated apparent clearance (CL/F) in our analysis is 0.080 L/h/70 kg. Considering a reported bioavailability of 47% after subcutaneous alemtuzumab, the absolute clearance would be calculated as 0.038 L/h/70 kg which is comparable to the clearance reported in a recent pediatric study (approximately 343 0.05 L/h/70 kg).

Pre-dose ALC values showed a trend of negative correlation with alemtuzumab clearance in our analysis but did not reach statistical significance. An earlier pediatric study also did not identify significant impact of baseline lymphocytes on alemtuzumab clearance.

However, we did find that alemtuzumab concentrations at Day 0 negatively correlated with pre-dose ALC(R²=0.68, P=0.0015), which is not surprising as high ALC counts should increase target-mediated drug disposition and high alemtuzumab concentrations should cause more lymphocyte depletion. Future larger-scale studies will help to further delineate the association between baseline ALC and alemtuzumab clearance. Alemtuzumab clearance may also be affected by the formation of anti-alemtuzumab antibodies. Careful attention to this will be required in the future.

The PK-PD relationship of alemtuzumab was evaluated using ALC counts as the PD marker in this study. Consistent with previous studies, we observed an immediate and almost complete ALC depletion after alemtuzumab treatment despite a wide range of initial ALC counts (0.06-6.52 k/μL). The developed population model includes an inhibitory Eₘₐₓ model. The estimated Eₘₐₓ of 1.27 k/μL equals the median value of baseline ALC, indicating the capability of a complete lymphocyte depletion. A relatively low mean EC50 of 0.062 μg/mL confirms a high potency of alemtuzumab to reduce the number of lymphocytes. For WBC depletion in adult patients with chronic lymphocytic leukemia, the estimated EC50 value is much higher (0.306 g/mL), which is not unexpected as CD52+ cells are only part of all WBC. Similar to the high variability associated with PK parameters, PD parameters also exhibit large inter-patient variability. The CV % for Eₘₐₓ was 85% and CV % for EC50 was over 100%. One of the limitations of this PK-PD modeling analysis is the lack of PK and PD data during the first 48 hours after the start of alemtuzumab treatment. In almost all subjects, ALC counts dropped to a minimum level within 48 hours, therefore the impact of alemtuzumab exposure on ALC count during this 48 hours interval could not be evaluated. However, this PK-PD model represents a first attempt to quantitatively describe the PK-PD relationship of alemtuzumab in children and young adults and provides a potential pathway for future development of a PD-guided dosing strategy for alemtuzumab therapy.

Currently, alemtuzumab is used off-label in pediatric patients and the dosing strategy is based on body weight (per kg). Our recent clinical study indicated that in almost half of the patients, a reduced alemtuzumab dosing of 0.5-0.6 mg/kg resulted in a Day 0 concentration above the target range. We further found that the current per kg dosing protocol for children and young adults may not accurately reflect the nonlinear relationships between body mass and alemtuzumab pharmacokinetics: in our pilot study, per kg dosing caused in general lower drug exposure in younger/lower weight patients and higher exposure in older/higher weight patients. We therefore considered allometrically scaled- and BSA-based dosing in addition to the per kg dosing in our clinical trial simulation study. Consistent with clinical observations, simulated alemtuzumab concentrations under per kg dosing were not equal across different age and body weight cohorts.

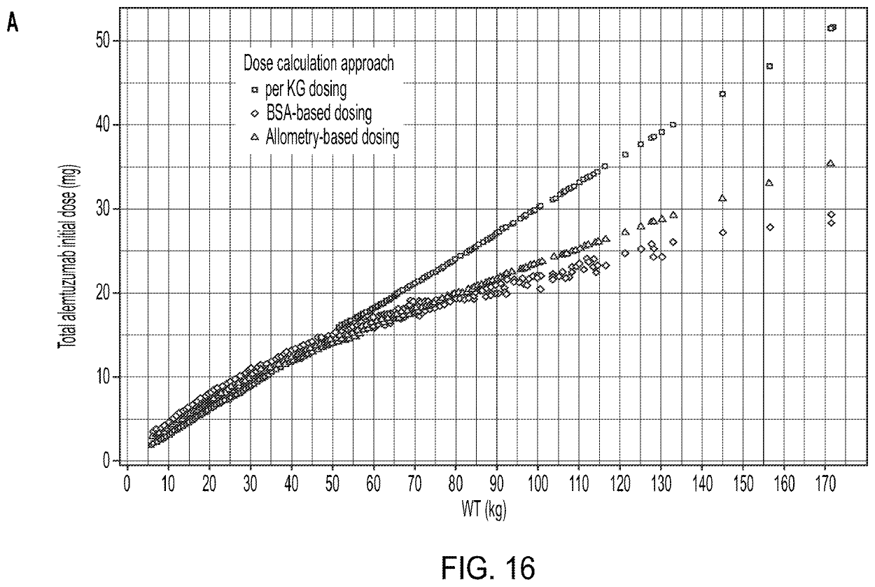

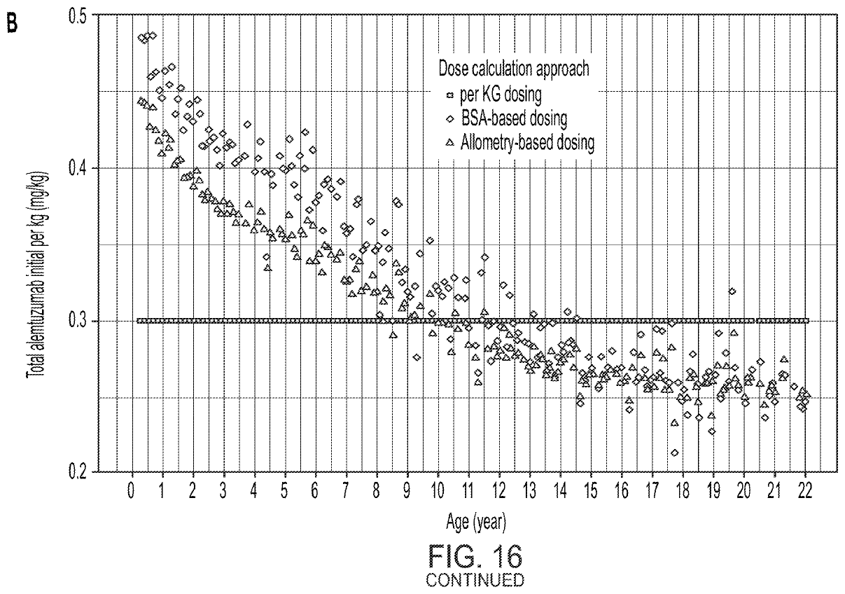

FIG. 16 illustrates the predicted dose levels with all three dose calculation approaches. As shown in FIG. 16A, compared to allometric dosing centered to 70 kg or BSA-based dosing, the linear per kg dosing would result in a slightly lower alemtuzumab dose for patients weighing less than 70 kg and a higher dose for patients weighing more than 70 kg. Because allometric dosing could accurately reflect the non-linear correlation between body size and clearance as identified in the population PK model, it has the potential to optimize alemtuzumab therapy for patients of all weights and ages. BSA-based dosing can also be considered for ease of clinical use given that allometric dosing is not widely used in clinical practice.

As indicated by the simulation analysis summarized in Table 4, 56.5% of the virtual patients achieved target attainment without the need for top-up doses under the BSA-based dosing (10 mg/m²), a notable increase to the previous reported 17% with a 0.5-0.6 mg/kg alemtuzumab dosing. It is worth noting that both BSA- and allometry-based dosing lead to higher per body weight (kg) doses in infants compared to in older children and young adults, as shown in FIG. 16B. In addition, the estimated doses according to BSA-based dosing are slightly higher in younger patients, and lower in the heavier (older) patients than the allometry-based doses. Therefore, we suggest to apply the allometry-based dosing to achieve the best target attainment in children of very young (<2 years) and the heaviest (>90 kg), though this consideration should be weighed against the standard clinical practice of dosing medications by body weight or BSA which are less likely to be associated with dosing errors.

The clinical outcome (efficacy and safety) of this newly proposed dosing scheme, however, remains to be confirmed. We plan to conduct a prospective clinical trial to further study target attainment with this new model-informed precision dosing scheme. For patients with a predicted Day 0 level below the target alemtuzumab plasma concentration (0.15 μg/mL), we propose to consider an average top-up dose (7 mg*(WT/70)0.75 (or 3.7 mg/m²) based on allometry or BSA, respectively) to bring the concentrations up to target. However, if Bayesian estimation are applied, a simulation can be conducted at the individual level and a precision top-up dose can be estimated for each patient similar to our previous study.

The developed PK model features a sequential zero- and 1^{t}-order absorption process to depict alemtuzumab subcutaneous administration. We understand that this model might be challenging to be implemented in clinical Bayesian forecasting software. Under this situation, a simplified model with a 1^{st}-order absorption rate constant of 0.0587 h⁻¹ and a lag time of 3.12 hours could be used as an alternative. This model also reasonably described the absorption process but is associated with a higher objective function value (ΔOFV=16) compared to the sequential zero- and 1^{st}-order absorption model.

The population PK-PD model of alemtuzumab after subcutaneous administration in pediatric and young adult transplant patients and simulation analyses suggest that an initial dose level of 18 mg*(WT/70)⁰-5 (or 10 mg/m²) divided over 3 days combined with a Bayesian adaptive dosing strategy would result in a better alemtuzumab target exposure attainment in pediatric and young adult patients undergoing allogeneic HCT. Further clinical studies are warranted to evaluate the efficacy and safety of this newly proposed dosing scheme.

1. A method of treating a non-malignant disorder in a subject comprising transplanting allogeneic hematopoietic cells into the subject; wherein the subject has a blood concentration of alemtuzumab in a range of about 0.15 μg/mL-about 0.6 μg/mL on day 0;
wherein day 0 is the day, or a planned day, when the transplanting of the allogeneic hematopoietic cells into the subject occurs.

2. The method of claim 1, comprising administering to the subject alemtuzumab starting at a day between day −22 and day −8; wherein day −22 is twenty-two days before day 0 and day −8 is eight days before day 0.

3. The method of claim 1, comprising administering to the subject alemtuzumab starting at day −14; wherein day −14 is two weeks or 14 days before day 0.

4. The method of claim 3, wherein the administering comprises administering alemtuzumab to the subject over two days starting at day −14, wherein the two days are day −14 and day −13: wherein day −13 is thirteen days before day 0.

5. (canceled)

6. The method of claim 3, wherein the administering comprises administering alemtuzumab to the subject over three days starting at day −14, wherein the three days are day −14, day −13 and day −12: wherein day −13 is thirteen days before day 0 and day −12 is twelve days before day 0.

7-11. (canceled)

12. The method of claim 1, wherein about 0.45 mg/kg-about 0.65 mg/kg alemtuzumab is administered to the subject; or wherein about 8 mg/m²-about 12 mg/m² alemtuzumab is administered to the subject.

13. The method of claim 12, wherein about 0.5 mg/kg-about 0.6 mg/kg alemtuzumab is administered to the subject; or wherein about 10 mg/m² alemtuzumab is administered to the subject.

14. The method of claim 12, wherein the alemtuzumab is administered to the subject over the three days of day −14, day −13 and day −12.

15. The method of claim 14, wherein if the subject weighs less than about 15 kg, the subject is administered about 0.2 mg/kg/dose of alemtuzumab on each of the three days.

16. The method of claim 14, wherein if the subject weighs equal to or more than about 15 kg, the subject is administered a test dose of about 3 mg of alemtuzumab on day −14.

17. The method of claim 16, wherein the subject is administered about a 0.2 mg/kg/dose to 0.3 mg/kg/dose on day −13 and about a 0.2 mg/kg/dose to 0.3 mg/kg/dose on day −12.

18. (canceled)

19. The method of claim 1, wherein the method further comprises determining alemtuzumab concentration levels in the subject before the first dose on day −14; determining alemtuzumab concentration levels in the subject 8 hours and 24 hours after administration of alemtuzumab on day −12; and determining alemtuzumab concentration levels in the subject daily until day 0.

20-29. (canceled)

30. The method of claim 1
the method further comprising:
administering to a subject alemtuzumab starting at day −14; wherein day −14 is two weeks or 14 days before day 0;
determining alemtuzumab concentration levels in the subject before the first dose on day −14; determining alemtuzumab concentration levels in the subject after administration of alemtuzumab on day −12; and determining alemtuzumab concentration levels in the subject daily until day 0;
modeling pharmacokinetic concentration time profiles of alemtuzumab between day −6 to day −4; wherein day −6 is six days before day 0 and day −4 is four days before day 0; and
predicting the alemtuzumab levels in the subject on day 0 using the pharmacokinetic modeling;
wherein if the alemtuzumab levels are predicted to fall below about 0.15 μg/mL on day 0, a top-up dose of alemtuzumab is given to the subject on day −3, day −2 or day −1; wherein day −3 is three days before day 0, day −2 is two days before day 0 and day −1 is one day before day 0.

31-46. (canceled)

47. The method of claim 30, wherein the pharmacokinetic modeling is carried out on day −5; wherein day −5 is five days before day 0.

48. The method of claim 30, wherein the top-up dose is given to the subject on day −3: or on day −2: or on day −1: wherein the top-up dose comprises about 0.2 mg/m² to about 5.0 mg/m² of alemtuzumab.

49-52. (canceled)

53. The method of claim 30, wherein the top-up dose comprises about 3.5 mg/m² to about 4.0 mg/m² of alemtuzumab.

54. The method of claim 1, wherein the alemtuzumab is administered intravenously or subcutaneously to the subject.

55. The method of claim 54, wherein the alemtuzumab is administered subcutaneously to the subject.

56. The method of claim 1, wherein the subject is a pediatric patient or a young adult patient afflicted with the non-malignant disorder.

57. (canceled)

58. The method of claim 1, wherein the non-malignant disorder is selected from immunodeficiencies, bone-marrow failure syndromes, inborn errors of metabolism (IEM) and hemoglobinopathies.

59-62. (canceled)
